SEQUENCE LISTING (105 sequences)

SEQ ID NO: 1 - DNA, 204 bp, Homo sapiens
ctatgaccttctgagtcttcttccatctgtgtgtgtatgtaagacagaaaaagagactgagagagagagagagagacaaagaacaggagaactacactgtggatttgggtgatgctccagggaggtttgaacagactgtcaatgtcatgttttgaagagagcaaggaattttggggatactgctgtgccagagaaaacaggttc

SEQ ID NO: 2 - DNA, 1043 bp, Homo sapiens
ccatcatcaatacaatattctgaattcattatttcacatcctaaacaaatctgttgatagcctgctaggcaatcacgaatcgtggtgctatgtaacattttattttgtgttctaagccacgaggaatcaagataaacctccaacacagccaggcccatactggaaagatctgcccttggcacacacaacagccagcagaggtcaccttccaagtggccctcagtggtggaaggagagcagttctggatggcagttctcatccttgtgctggccttgtcttctgtccagcaataaagctttaggctttgctttctccatttgccgtggaggagagtaacagtcacagcagtaatcaagacaggcgtcttgtcatttaattaagcatggcatccctagtatccataatttcaggtcattaggtacccagacattgttctctccattttgcagatgaggaaacaaaagcccagggaaatagtatacttgcccagaaagaatttaaaaaaatattgttgagcaggctagcgtctgtgggtgcagttcctctgattcagtgtgtcatgccgcaaacatgccaggcacattcccacctcagggcctttgcactggctgtttgcatccattggcctttgcattgcaggaaacactcttaccccaggtaccagcatggctcattccaccactaggttcccatgttaaaaccaggcaagtctaggacaaactaggacgagttggtatacagtagaccagtaaatgaaagagcttgtgttatggagctgacattcttgttagggatgctcacctggcccatctcatttaaaattgcactcttccctcactggctggacgaggacggtctctcctgggattgcttggctcagctccacatggtcgctcatcctcctgggagctcatctgggcttgttctcatggcagaagcagagcagaagcgtgcaagcttcttgggacctaggcacaccatccctaatgtgacacgatgttgcctggagttgaggagtggagaaaaagtctccagatcttga

SEQ ID NO: 3 - DNA, 756 bp, Homo sapiens
atttgtttaaaacaattattgatttctaaagtggcaccacctaagttaccaaatctttattaagaatttacaatatgttctatacggtatggggtaccaagtttagctcagttgctgttaatgaagtgtctaccatgggttagacacagtcactaggtaccagggtgtgaatgccccagcatgtggctggctttgagattttatcatctcattagagtaaaggacttgtaaggggcaaggtgaagttgccttttaatatggtaagtgcaaatatcaggttagtacaggagccagggggagcacaaggaggtcagtttcccagactaagaggagtggggagtatgttcagggagggcctcctggaagagccaatatccgatctgcaggggttatatcacctgctgttttctcagaacttcccattgttctgagctcattgtttgttctctcccaaaagaaattacatcaggacaatatttctatcctgaacaaacacttttatcttctgactcatttatggaaggagatcctttctttgattatttgtgtacatactttgacttgttcttctcttgttcccaactccattttttcctgtttcctgcccaccagcctcatatacacacacatttgcatttaagacctgtgaaaacaaggactattgacttcatttttgtatctacaaccaaagccttgaaaatagtatgtactctgtacatattcattaaatttaatgtatttactgcaaaaaaaaaa

SEQ ID NO: 4 - DNA, 1931 bp, Homo sapiens
caattattgtacccagaaatagccacatgacttacatccttacctccttcagatcctcgctaaattgtcatcctctcacccatgcctttttccctgaccagcctgttttaaattgacactgttttcactgctaaatctcttagcacttcctgtctcttcattacttttttttttaaaatcaccatctgatacatattttacttgagctttgttatgggatacagttaagctactagaaacaattaattcaaagataagcttaattcattggtgcgcttcacaagggtaggaatttttgcgtgttttgttcacagtggtgaatccccagcacctagaacaatgccaggaaaaacaaccactcaataaatgctggtttaatgaaaaagaaaaatcttaaaaaccaagtccaccatatatatcccacaaagataggaaagtgggattggggaaagggatgctatagtagtttgactttgcttattacacccttctctagttgctgactgatcatagaattgggtaaatggcttgagctgggacaatttattgtaaatataaagcactaaggcaaatataaaggattggaacttcgtactgagaggacctctgcttccagccatcttgtaataacaggaactggagttattctctcgcctaaaatagcctaacaaaccgacacacacaaaaagcccataaaacatatgaaacaaaggcattccgacattggacaagtggcacgagacagtgaaccctgagagaaggaaaaaacaaatgatttgccccaggctgcagtgcaggaaggcagaacccaaacacagcttatcctaggttgagatgatggagttaggaattctgggaggctgaggcagctagaatttgaaggacagagtaccagagaagagagagttagttacacagagagtgagctccagagatcttcagagggctctcctggaggcttcagccgagtgctgattagtgcatatgcataagagaactacccatggatgaggaaagaggcactgggaagaagaaagtagctcatataaggcagtgaagagtgtgtcccccccaccccacccccaccattcagagtggaaaaaccttgtatataagtgggattagatggtgtactcagaagactaatgcttcagcgatgaggcaaaattagccctagactagtctaatctgatcctgcctaaaaaatcttaaaagtaagccattcaagacaggtcataaaactgttttgtaaagggccagatggaaaatattttaggatatgtgggccatgtagtctctgttttaactactcacttctgcccttctagtgttaaaacagccataaacaatttgtaagcaaatgtatttacatgtatcagaatgtattccaataaaattttatttatagaaataggcagtggtccagatttggcctgtgggcctagtttgcagacacttggtttgaaggatcaaattgtttgaagtagcttaactgtatcccagaacaaagctcaaggatatttaaggaaatacaaaatatccagcacccattaataaagtatgcaatatgtagcattcagtcagaatttaccagatgtgcagagaagcaggaaaattcaacagtgaagagaaaactcaatcaatagaaacagacccagaagtgacacacatctaataagtggaggtactaaaataaattaaatagatttaataaggatattcaaatagttattgttactatgttccatatactcaagatggtagtggaaagcataaggtggttaagaaaagatggaagctataaaaagacctaaattgaacttctagacctctagacattttataaacacctgagttgaaaaatacactggataggattaaaatcccaaaatacactgcatagcaattatacaaaatgagatactgaatgg

SEQ ID NO: 5 - DNA, 2276 bp, Homo sapiens
ctagaatcagcaatgtatttatttgtttggtgtttacgtaagtcttagtgtgttttataaataataggagggtttgtttaaagtgggcttttgaagatacagtgtgactttaaaagaatggtcttagaaaacttataaatagcctaaatatgtactgaagaacatttcataatcacaaaaaaagaaataatatatctcatgaattattaagaatttgttttttaaataaagtcctctttttgaaaaggtattaagaagaggaattagacaaaacttgtttgaatatggttgtaattgcagtttactgagttatatataaaaaagcccaattagtttaaacaaatacagtatctgggtttatggtattctgagcctcgtattttttcattgaatgtcaggggtatattaaaatgcctccctttatgtttctatacaaaagattgttctgacagtaagtttaagtgattttcacaacttattgctgtctaaaaatttcccttgacttcacattttgttctgaatatgttggtttctttcctccacccacacaatgttgaaggcaagggaagcaagagttgcaaagtaatacagtgaatttgagaaccctggctttttttcaaaacaaaataaacctttattataagattgctttttattgataccaaaaaagggtggcactttggcaaatggtcagtaagtgtatgaatagcctaattgatttagctggaatgaataagaagctttgctatttgtccttgtagatagttggctcagtaaacattgtgatggtgatagaccaaattttccatggtgaaaggtaaaacagaacagcaacaaaaaactcatcaaacttatagatccaaatatcagcaattttgtgtctgtgtcaaggtcttcaaagaaggagcaatgtgttaattaggatctcatttgaatttctatagtgaaaaaataatcatgttaacttacataaaacccaagagtttgacaaatgttactatgtgaaaatattaatagaggagatccttgctgagaatttaaatctgcatgaaattcatagatgtgtgtagaaatttaaatttatcatacactccgcagtaaatgccaataaaggtggatggcataattaggacaaagataacagtagaagcaaaataagaattttttaaaaaccttatggatgtcacattagtctgactcagtctgtaactctagttagaaatgtaaaattttagggatttttgggctactgtaagaaaagaatctgctttgggggtgggatgtgaggagaaagaaatgtttgtggcaattaagatgtatatagtatatttaagaagtacatttgcaattctaatcacttagttcaaaggagaaaaatatatcatgtgtaactttatttacttttaatactattattattattacatgttgaaaatagaaaatcttgaaaatttctagagggtaaaagtgttatttctaattccattagctccttttaagataggaacatttcatattagtctccctagcacagaatctgaaccataataggcactccataaatgtttactgaactaacctgaattttagaaatatcagggtgcaaagcagaattatttttctaggctttaaaactataccactgctccttacccctgtacacaccttcgaatctcagttttgatacaaataggttctcagttttgggtgtgaagaaaactggttgatatttttttttaatctactaaatgttggccagagctgaaattgcctgaaagggaacttgatttagcagaattccttcttttcagaaagaaggacttagactaaattatatgaacgggaggagcctgtttttatctgagatggttgattggaaaaagaatagataaactaaggtggagagttgatttgaaaaaaggaaaaaagatagtcctgtctagtggtcagtccctaacagtgtggttaaaggggagctaaatttcaactttcaggggtcagctgagacaaccacctagattttccaagttagttccaatttcaagtacaataatcctttgacaacatagaaaagaattatagttagcaaaattgcaattgtcacagtcaaggtcttgttggtaatagagggttggggaaaaagttaaaacagcattaaatgaatatatttttaacataacaaaagttcccagaagacatccacttcaaattcaagccatattcatcaatgttaaaaaccttactcttctccctgatagtgtgaggaaaaataaaat

SEQ ID NO: 6 - DNA, 183 bp, Homo sapiens
ctgaggagatcaagcaggggcaggcaacaggaatcctagaatggcccttttggactaaattggtggttgtggccatcggcttcaccggaggacttctttttatgtatgttcagtgtaaagtgtatgtgcaattgtggaagagactcaaggcctataatagagtgatctatgttcaaaactgtc

SEQ ID NO: 7 - DNA, 259 bp, Homo sapiens
ggcattttacacacattggtacttgctaattgctattccaagaaatgaacaaaggaacaaaagaaatgaacaaaagaaacattttctttggctgagttatattttaatattatgcatttcagtgagaactgacattagaagtagctatataaaaggccatcatagttgtaaactattgttgtaaactatagttaaagacattgtccagtatgttgactggcattcttggagtaaattcctcctgtgatttaatacttgg

SEQ ID NO: 8 - DNA, 534 bp, Homo sapiens
ccttataaagtatgcacagcacttatatctattctagaacttggggatggaggagatatgtttgattaaataatgaaccatgagagggtctaatgaagcttggccaagaagtattttgctcagatagttcatgctatatcttattgccataaactccatgtggttcacagagacttaaaccagagaatgtagtcttctttgaaaacaaggtcttgtaaagttgacagactttgggttcagcacaaatttcaaccagggaagaagctcactacaagctgtggatctcttgcatattccgctccagaaattctgcttggtgatgagtatgatgcacctgcagtagatatttggagtctgggagtgatccttttcatgttggtgtgtgggcagccgccctttcaagaagccaatgacagtgaaacactgacaatgatcatggattgcaaatatacagtaccatcccatgtgtctaaagagtgtaaagacctaatcacacggatgctacagagagatcccaagagaagggcttcttta

SEQ ID NO: 9 - DNA, 500 bp, Homo sapiens
tgaaaggcaaaacaaacctgtcagacctgtttcaccacattgcaatcaatactccttgtctatacaaattttctgctcaccacaccaacgttcatttgctgatcagattctagaatatgtgcacctatgtgtcagaatgaagccgtggtctctcccacactttgaggaaatggacatcattgctgacatacacttgatagaaaatgttagttctagctaggtgttcaaaactctagcaagtaaataggaatgcactgtttttgtttgttttttatgtttttatcattatatttggagaattttctgaagtagctctttttttaaaacagctttattgaggtatagttgaaatgcaaaagaggcacatatttaatatatacagtttaatgagtttggatgttattggggacagctgttgtgaaacttcagttcttgtcttctcagtttaaaagaagcaagagacacacagcaaaggagatgcagtgtagagtaatttattg

SEQ ID NO: 10 - DNA, 410 bp, Homo sapiens
ctaaacctcaaaatataactatttacatctcatttgctatttgtttccttgtttttagtatacatctatttaaaaatgtgtttatttcagtttaactgtagaagttttaagaacaaaaggcccttgtgttgttgaagaaaatgaccccatatttgagcgcggttctacaactacatactccagctttaggaaaaactattactctaaaccatggtcaaataaaggtaactaattttcatttaaaaatgtgtaagttctctatttgaaatgaattgactgtttctgaattaaatcagttctcccctaagtacataaaatgcaaactaccattttattccagctaattttattgacacctctatcttatgccttccatggaatggtatctctgactttgtcttacaaatctt

SEQ ID NO: 11 - DNA, 293 bp, Homo sapiens
gaactgctttagaaggaccagaatcaacactgaaaacaaatcagaggtaaagagggtaggcatgagaagaaaccacagagaaggcagtggaaaaatacaaggcaatggaagaaattaactaggaactcagatgtggagagacaagttgggaatgatttgtgtactgggagatgaaaaactaaaatgagctggagctaaataatgcatcaatattttataaatatactgttatccaaaacaaaacaaaacaaatagcagaagaaggaatgcaagtttttatcctttatgtaaag

SEQ ID NO: 12 - DNA, 294 bp, Homo sapiens
cattaggcacctccaaagggacaaaggaccaatatacctggttggggacaggattctgtcatttgattattcctgactcatgttttcatgaggtagtcccccacctcatataaaagcctcagtgttggcttctgaccatggtgtatgaaaagcccttgtctaaaggttactgccctgagaaaataataaaggaagaagaggatagacatgaagacactttaaagcctcctgaatagaatgcatccagaagcgaattccaggagattctgtcatcatgcttgcctttcaagcaaa

SEQ ID NO: 13 - DNA, 301 bp, Homo sapiens
cagtaccttggcttggctcttgacgtggacagaattaaaaaggaccaagaagaggaagaagaccaaggcccaccatgccccaggctcagcagggagctgctggaggtagtagagcctgaagtcttgcaggactcactggatagatgttattcaactccttccagttgtcttgaacagcctgactcctgccagccctatggaagttccttttatgcattggaggaaaaacatgttggcttttctcttgacgtgggagaaattgaaaagaaggggaaggggaagaaaagaaggggaagaagat

SEQ ID NO: 14 - DNA, 247 bp, Homo sapiens
gcaggcctgacccctgggataggtcagggcggtggttccttgggagaattcctgcttgatgagatggaaggtccaagtcaatagcctcatggtcctcccaagtctgacagtctgctattctacacacctgtccacaggctgcagacatataaaggtaaatgttcaggtattagaaaatattcaaagaattctcaatgttcaaaattctgaaaagcaaatctatgctgaatgtgtggtgggggcattc

SEQ ID NO: 15 - DNA, 320 bp, Homo sapiens
caagtagtagtggaatgggagttgttgcttgtattagttctggtagttcaatatgaacatctagagtatatctctaagaacaaagagtttaattctgccatttgggctagttcttgtagagaaatacagtgagagaatctgagatatcctcttccccaaataatttataattcataagggggaaataaaccttacacagaacttttgcgatatagtaatgcctcacatttaacaaacatagtttttcattcccatttttacatttaatgcttatttaaaaatctcatcaaatagttaagagaccagtgtcattcttattt

SEQ ID NO: 16 - DNA, 757 bp, Homo sapiens
acatacaactggttttgcaggtgagagtcactttgtaacatgtttcaacaccttcctctcctgataagcagtttctagtctgaatcaggactttaagaatatgctgtataatgtttgtaccaatacgaagcaagtgttttcctctctcacagccttttttgtgatgattagaaacttaccctctgggccattcctcctgaaattgccacctccagaccaccaatcttttaaaaatgtaaggctaattataagcgtattttcacctacaatgtgcgtgtgcgtgtgtgccatgtgtgcacgtacatgaatagctgtaccaggtaggagatctctcacacttggctggacactccagctacgcgggaacaagcacctgagcggggtcctcaaaaccaaccaggattccgcccgccgacgtcctggctgtgactggctttgggaagtttgactccatgccatcctttgcttctccactcggttctgatgaagaaaactgaattgcgcttccacaattcagtggcaggagagggtggacctctcgagttcccaagggccccctcctgtcacctgccccagagccccatgcagggccacactgacccccagggaaagctgacactagtagagtcagctgttaaagggtaatagaggtcaaaggctgggtagagggtttttagtttttgtgtttttacacacagagaaaatgagactaatcactggtttataatagatcaggagactatcatttcttttgttc

SEQ ID NO: 17 - DNA, 197 bp, Homo sapiens
gagttatctttcccaccagaaagctgaaattgctaggagaaggtccagttctgataagcaccaaagcccaggctcttgcaacacctcctcatgtgcctgattctacctttgtcctgataccatcagccagagccttctctaacacagtgatgtttcctccttgttacctttgcgcgccgtcctgaaaatatccattc

SEQ ID NO: 18 - DNA, 17 bp, Artificial Sequence
  Synthetic
gttttttttttttttta

SEQ ID NO: 19 - DNA, 17 bp, Artificial Sequence
  Synthetic
gtttttttttttttttc

SEQ ID NO: 20 - DNA, 17 bp, Artificial Sequence
  Synthetic
gtttttttttttttttg

SEQ ID NO: 21 - DNA, 10 bp, Artificial Sequence
  Synthetic
ttgatggacc

SEQ ID NO: 22 - DNA, 10 bp, Artificial Sequence
  Synthetic
tattgccgtg

SEQ ID NO: 23 - DNA, 10 bp, Artificial Sequence
  Synthetic
gacggttagt

SEQ ID NO: 24 - DNA, 10 bp, Artificial Sequence
  Synthetic
ggacttcgta

SEQ ID NO: 25 - DNA, 10 bp, Artificial Sequence
  Synthetic
ggaagagttg

SEQ ID NO: 26 - DNA, 10 bp, Artificial Sequence
  Synthetic
atccgtactg

SEQ ID NO: 27 - DNA, 10 bp, Artificial Sequence
  Synthetic
tccctacaga

SEQ ID NO: 28 - DNA, 10 bp, Artificial Sequence
  Synthetic
tcatagtccg

SEQ ID NO: 29 - DNA, 10 bp, Artificial Sequence
  Synthetic
aaacccatcg

SEQ ID NO: 30 - DNA, 10 bp, Artificial Sequence
  Synthetic
tattgccgtg

SEQ ID NO: 31 - DNA, 10 bp, Artificial Sequence
  Synthetic
cagaactgct

SEQ ID NO: 32 - DNA, 10 bp, Artificial Sequence
  Synthetic
aaggaacgga

SEQ ID NO: 33 - DNA, 10 bp, Artificial Sequence
  Synthetic
atgggaggaa

SEQ ID NO: 34 - DNA, 10 bp, Artificial Sequence
  Synthetic
gctggttttg

SEQ ID NO: 35 - DNA, 10 bp, Artificial Sequence
  Synthetic
gtttgcttgc

SEQ ID NO: 36 - DNA, 10 bp, Artificial Sequence
  Synthetic
cacgagtcta

SEQ ID NO: 37 - DNA, 10 bp, Artificial Sequence
  Synthetic
gagcaaggta

SEQ ID NO: 38 - DNA, 24 bp, Artificial Sequence
  Synthetic
tgttttctctggcacagcagtatc

SEQ ID NO: 39 - DNA, 22 bp, Artificial Sequence
  Synthetic
tccagggaggtttgaacagact

SEQ ID NO: 40 - DNA, 32 bp, Artificial Sequence
  Synthetic
ccaaaattccttgctctcttcaaaacatgaca

SEQ ID NO: 41 - DNA, 26 bp, Artificial Sequence
  Synthetic
tccctagtatccataatttcaggtca

SEQ ID NO: 42 - DNA, 20 bp, Artificial Sequence
  Synthetic
cctgggcttttgtttcctca

SEQ ID NO: 43 - DNA, 33 bp, Artificial Sequence
  Synthetic
aggtacccagacattgttctctccattttgcag

SEQ ID NO: 44 - DNA, 22 bp, Artificial Sequence
  Synthetic
tttcccaactaagaggagtggg

SEQ ID NO: 45 - DNA, 22 bp, Artificial Sequence
  Synthetic
gatataacccctgcagatcgga

SEQ ID NO: 46 - DNA, 26 bp, Artificial Sequence
  Synthetic
agggagggcctcctggaagagccaat

SEQ ID NO: 47 - DNA, 20 bp, Artificial Sequence
  Synthetic
cgacacacacaaaaagccca

SEQ ID NO: 48 - DNA, 21 bp, Artificial Sequence
  Synthetic
ttcactgtctcgtgccacttg

SEQ ID NO: 49 - DNA, 32 bp, Artificial Sequence
  Synthetic
aaaacatatgaaacaaaggcattccgacattg

SEQ ID NO: 50 - DNA, 24 bp, Artificial Sequence
  Synthetic
ccatggaaaatttggtctatcacc

SEQ ID NO: 51 - DNA, 24 bp, Artificial Sequence
  Synthetic
gctggaatgaataagaagctttgc

SEQ ID NO: 52 - DNA, 36 bp, Artificial Sequence
  Synthetic
tcacaatgtttactgagccaactatctacaaggaca

SEQ ID NO: 53 - DNA, 17 bp, Artificial Sequence
  Synthetic
gtcctccggtgaagccg

SEQ ID NO: 54 - DNA, 21 bp, Artificial Sequence
  Synthetic
acaggaatcctagaatggccc

SEQ ID NO: 55 - DNA, 25 bp, Artificial Sequence
  Synthetic
tggccacaaccaccaatttagtcca

SEQ ID NO: 56 - DNA, 28 bp, Artificial Sequence
  Synthetic
atagtaccatcaagcaactggatagagt

SEQ ID NO: 57 - DNA, 25 bp, Artificial Sequence
  Synthetic
tttgtctgtcttgaggaatgtttca

SEQ ID NO: 58 - DNA, 35 bp, Artificial Sequence
  Synthetic
caacaacagcgattacatattctcctcaagcctac

SEQ ID NO: 59 - DNA, 18 bp, Artificial Sequence
  Synthetic
ggcttcttgaaagggcgg

SEQ ID NO: 60 - DNA, 24 bp, Artificial Sequence
  Synthetic
cagtagatatttggagtctgggag

SEQ ID NO: 61 - DNA, 25 bp, Artificial Sequence
  Synthetic
tgcccacacaccaacatgaaaagga

SEQ ID NO: 62 - DNA, 24 bp, Artificial Sequence
  Synthetic
gcacctatgtgtcagaatgaagcc

SEQ ID NO: 63 - DNA, 28 bp, Artificial Sequence
  Synthetic
ttctatcaagtgtatgtcagcaatgatg

SEQ ID NO: 64 - DNA, 28 bp, Artificial Sequence
  Synthetic
tggtctctcccacactttgaggaaatgg

SEQ ID NO: 65 - DNA, 20 bp, Artificial Sequence
  Synthetic
gggcccttgtgttgttgaag

SEQ ID NO: 66 - DNA, 31 bp, Artificial Sequence
  Synthetic
atagtttttcctaaagctggagtatgtagtt

SEQ ID NO: 67 - DNA, 27 bp, Artificial Sequence
  Synthetic
atgaccccatatttgagcgcggttcta

SEQ ID NO: 68 - DNA, 22 bp, Artificial Sequence
  Synthetic
tccattgccttgtatttttcca

SEQ ID NO: 69 - DNA, 26 bp, Artificial Sequence
  Synthetic
gaaaacaaatcagaggtaaagagggt

SEQ ID NO: 70 - DNA, 28 bp, Artificial Sequence
  Synthetic
tgccttctctgtggtttcttctcatgcc

SEQ ID NO: 71 - DNA, 24 bp, Artificial Sequence
  Synthetic
ggcagtaacctttagacaagggct

SEQ ID NO: 72 - DNA, 22 bp, Artificial Sequence
  Synthetic
gttttcatgaggtagtccccca

SEQ ID NO: 73 - DNA, 26 bp, Artificial Sequence
  Synthetic
ccatggtcagaagccaacactgaggc

SEQ ID NO: 74 - DNA, 24 bp, Artificial Sequence
  Synthetic
tgcataaaaggaacttccataggg

SEQ ID NO: 75 - DNA, 27 bp, Artificial Sequence
  Synthetic
ggatagatgttattcaactccttccag

SEQ ID NO: 76 - DNA, 26 bp, Artificial Sequence
  Synthetic
tggcaggagtcaggctgttcaagaca

SEQ ID NO: 77 - DNA, 21 bp, Artificial Sequence
  Synthetic
gggaggaccatgaggctattg

SEQ ID NO: 78 - DNA, 19 bp, Artificial Sequence
  Synthetic
cggtggttccttgggagaa

SEQ ID NO: 79 - DNA, 28 bp, Artificial Sequence
  Synthetic
cttggaccttccatctcatcaagcagga

SEQ ID NO: 80 - DNA, 27 bp, Artificial Sequence
  Synthetic
aaaactatgtttgttaaatgtgaggca

SEQ ID NO: 81 - DNA, 27 bp, Artificial Sequence
  Synthetic
cctcttccccaaataatttataattca

SEQ ID NO: 82 - DNA, 38 bp, Artificial Sequence
  Synthetic
actatatcgcaaaagttctgtgtaaggtttatttcccc

SEQ ID NO: 83 - DNA, 21 bp, Artificial Sequence
  Synthetic
cagaaccgagtggagaagcaa

SEQ ID NO: 84 - DNA, 19 bp, Artificial Sequence
  Synthetic
cgacgtcctggctgtgact

SEQ ID NO: 85 - DNA, 27 bp, Artificial Sequence
  Synthetic
atggcatggagtcaaacttcccaaagc

SEQ ID NO: 86 - DNA, 17 bp, Artificial Sequence
  Synthetic
agcaccaaagcccaggc

SEQ ID NO: 87 - DNA, 23 bp, Artificial Sequence
  Synthetic
gatggtatcaggccaaaggtaga

SEQ ID NO: 88 - DNA, 26 bp, Artificial Sequence
  Synthetic
cttgcaacacctcctcatgtgcctga

SEQ ID NO: 89 - DNA, 25 bp, Artificial Sequence
  Synthetic
tcacccacactgtgcccatctacga

SEQ ID NO: 90 - DNA, 25 bp, Artificial Sequence
  Synthetic
cagcggaaccgctcattgccaatgg

SEQ ID NO: 91 - DNA, 26 bp, Artificial Sequence
  Synthetic
atgccctcccccatgccatcctgcgt

SEQ ID NO: 92 - DNA, 28 bp, Artificial Sequence
  Synthetic
aggagagtaacagtcacagcagtaatca

SEQ ID NO: 93 - DNA, 24 bp, Artificial Sequence
  Synthetic
ccctgggcttttgtttcctcatct

SEQ ID NO: 94 - DNA, 26 bp, Artificial Sequence
  Synthetic
ggtcagtttcccaactaagaggagtg

SEQ ID NO: 95 - DNA, 25 bp, Artificial Sequence
  Synthetic
ggaagttctgagaaaacagcaggtg

SEQ ID NO: 96 - DNA, 19 bp, Artificial Sequence
  Synthetic
acattggacaagtggcacg

SEQ ID NO: 97 - DNA, 24 bp, Artificial Sequence
  Synthetic
ccatggaaaatttggtctatcacc

SEQ ID NO: 98 - DNA, 24 bp, Artificial Sequence
  Synthetic
gctggaatgaataagaagctttgc

SEQ ID NO: 99 - DNA, 21 bp, Artificial Sequence
  Synthetic
acaaatcagaggtaaagaggg

SEQ ID NO: 100 - DNA, 21 bp, Artificial Sequence
  Synthetic
tgatgcattatttagctccag

SEQ ID NO: 101 - DNA, 162 bp, Homo sapiens
gaggagagtaacagtcacagcagtaatcaagacaggcgtcttgtcatttaattaagcatggcatccctagtatccataatttcaggtcattaggtacccagacattgttctctccattttgcagatgaggaaacaaaagcccagggaaatagtatacttgcc

SEQ ID NO: 102 - DNA, 170 bp, Homo sapiens
tacaggagccaggggggagcacaaggaggtcagtttcccaactaagaggagtgggggagtatgttcagggagggcctcctggaagagccaatatccgatctgcaggggttatatcacctgctgttttctcagaacttcccattgttctgagctcattgtttgttctctcc

SEQ ID NO: 103 - DNA, 158 bp, Homo sapiens
ctgagaggacctctgcttccagccatcttgtaataacaggaactggagttattctctcgcctaaaatagcctaacaaaccgacacacacaaaaagcccataaaacatatgaaacaaaggcattccgacattggacaagtggcacgagacagtgaaccc

SEQ ID NO: 104 - DNA, 256 bp, Homo sapiens
ttgctgttctgttttacctttcaccatggaaaatttggtctatcaccatcacaatgtttactgagccaactatctacaaggacaaatagcaaagcttcttattcattccagctaaatcaattaggctattcatacacttactgaccatttgccaaagtgccacccttttttggtatcaataaaaagcaatcttataataaaggtttattttgttttgaaaaaaagccagggttctcaaattcactgtattactttg

SEQ ID NO: 105 - DNA, 225 bp, Homo sapiens
gataacagtatatttataaaatattgatgcattatttagctccagctcattttagtttttcatctcccagtacacaaatcattcccaacttgtctctccacatctgagttcctagttaatttcttccattgccttgtatttttccactgccttctctgtggtttcttctcatgcctaccctctttacctctgatttgttttcagtgttgattctggtccttctaa

(19) United States
(12) Patent Application Publication
(10) Pub. No.: US 2004/0058351 A1
(43) Pub. Date: Mar. 25, 2004

(54) Method of examining for allergic disease

(76) Inventors: Yuji Sugita, Ibaraki (JP); Ryoichi Hashida, Ibaraki (JP); Kaoru Ogawa, Ibaraki (JP); Takeshi Nagasu, Ibaraki (JP); Hirohisa Saito, Tokyo (JP); Masaya Obayashi, Tokyo (JP)

Correspondence Address: Peter G Carroll, Medlen & Carroll, 101 Howard Street, Suite 350, San Francisco, CA, 94105, US

(21) Appl. No.: 10/398,877

(22) PCT Filed: Sep. 28, 2001
(86) PCT No.: PCT/JP01/08574

(30) Foreign Application Priority Data
Oct. 13, 2000 (JP) 2000-314093

(51) Int. Cl. C12Q 1/68; C07H 21/04

(52) U.S. Cl. 435/6; 536/24.3

(57) ABSTRACT

The differential display method was used to search for a gene whose expression level in eosinophils of patients with atopic dermatitis. As a result, 17 genes showing a significant increase in expression in eosinophils of light patients were isolated. These gene are usable in testing for an allergic disease and screening for a candidate compound for a therapeutic agent therefor an allergic disease.

### TECHNICAL FIELD

The present invention relates to genes associated with early stage allergic diseases, methods of testing for allergic diseases and methods of screening for compounds that serve as candidate therapeutic agents for allergic diseases using the expression of the genes as an indicator.

### BACKGROUND ART

Allergic diseases, such as atopic dermatitis, are considered to be multifactorial diseases. These diseases are caused by the interaction of many different genes, whose expressions are influenced by various environmental factors. Thus, the determination of specific genes causing a specific disease has been extremely difficult for allergic diseases.

Additionally, expression of mutated or defective genes, or overexpression or reduced expression of specific genes is thought to be involved in allergic diseases. To elucidate the role of gene expression in diseases, it is necessary to understand how a gene is involved in triggering disease onset and how the expression of the gene is altered by external stimulants, such as drugs.

Recent developments in gene expression analysis techniques have enabled analysis and comparison of gene expression in many clinical samples. Among these methods, the differential display (DD) method is useful. The differential display method was originally developed by Liang and Pardee in 1992 (Science, 1992, 257: 967-971). According to this method, several tens or more different samples can be screened at one time to detect genes whose expressions differ among the samples. Important information vis-à-vis causative genes of a disease is expected to be uncovered through examining genes with mutations or genes whose expression changes depending on time and environment. Such genes include those whose expression is influenced by environmental factors.

History taking, and confirmation of family history and anamnesis of the patient are important in general for recent diagnosis of allergic diseases. Further, methods of diagnosing allergy based on more objective information include a method in which patient's blood sample is tested and method of observing patient's immune response to allergen. Examples of the former method include the allergen-specific IgE measurement, leukocyte histamine release test, and lymphocyte stimulating test. The presence of allergen-specific IgE verifies the allergic reaction against the allergen. However, allergen-specific IgE is not always detected in every patient. Furthermore, the IgE assay requires performing tests for all of the allergens necessary for diagnosis. The leukocyte histamine release test and lymphocyte stimulating test are methods for observing the reaction of the immune system toward a specific allergen in vitro. These methods require complicated operation.

Another known method of allergy diagnosis is based on the immune response observed at the time when a patient is contacted with an allergen (example of the latter method). Such tests include the prick test, scratch test, patch test, intradermal reaction, and induction test. These tests allow for the direct diagnosis of patient's allergic reaction, but are regarded as highly invasive tests because patients are actually exposed to allergen.

In addition, regardless of the allergen types, methods to testify the involvement of allergic reaction are also attempted. For example, a high serum IgE titer indicates the occurrence of allergic reaction in a patient. The serum IgE titer corresponds to the total amount of allergen-specific IgE. Though it is easy to determine the total amount of IgE regardless of the type of allergen, the IgE titer may be reduced in some patients, for example, those with non-atopic bronchitis.

The number of eosinophils and eosinophil cationic protein (ECP) value are used to diagnose delayed-type reaction following Type I allergy and-allergic inflammatory reaction. The number of eosinophils is considered to reflect the progress of allergic symptoms. ECP, a protein contained in eosinophil granules, is also strongly activated in patients with an asthma attack. Even though these diagnostic items reflect allergy symptoms, in reality, the general observation is that increase of eosinophils becomes clearly noticeable with advancement of allergic symptoms. That is, the period in which an increase in eosinophils is clearly observed is often accompanied by marked allergic symptoms. Therefore, the number of eosinophils cannot be used as an indicator for the early stage of an allergic disease.

Therefore, a marker for an allergic disease that is not dangerous towards patients and that can allow for easy acquisition of information necessary for early diagnosis, would be useful. Since such a marker is considered to be deeply involved with the onset of an allergic disease, it may be an important target not only for diagnosis but also for the control of allergic symptoms.

### DISCLOSURE OF THE INVENTION

An objective of the present invention is to provide novel genes that can be used as allergy indicators, particularly for early stage allergic diseases. Another objective of the present invention is to provide methods of testing for early stage allergic diseases, and methods of screening candidate compounds for therapeutic agents for allergic diseases, both methods using the indicators.

The genes associated with early stage allergic diseases are located upstream of other genes associated with allergic symptoms, and may play the role of inducing the expression of these other genes. The present inventors hypothesized that if such genes could be identified, investigating their expression state would allow diagnosis of early stage allergic diseases.

Furthermore, the present inventors thought that such genes could also be used as important targets in treatment of allergic diseases. Effective pharmaceutical agents for early stage allergic diseases may be used as effective therapeutic agents against fundamental causes of pathology, not only at early stages of allergy but also after advancing to severe conditions. Pharmacological effects leading to complete cure of allergies instead of a mere symptomatic treatment can be expected from such therapeutic agents.

First, the present inventors isolated genes whose expression differed between the peripheral blood eosinophils obtained from healthy subjects and patients with atopic dermatitis. The differential display (DD) system (WO 00/65046) developed by the present inventors was applied to the method for obtaining a gene based on differences in expression levels. This a DD system is based on the previously established procedure of “Fluorescent DD method” (T. Ito et al., 1994, FEBS Lett. 351: 231-236), wherein leukocyte RNA samples prepared from the blood of a plurality of humans are analyzed. Eosinophils were selected as the target cells for the gene expression comparison. Eosinophils are important indicators for allergic symptoms. Therefore, genes whose expression levels differ in eosinophil cells are considered to be closely related to allergic symptoms.

Next, the present inventors compared the expression levels of genes obtained by the DD system in patients at different stages of advancement of allergic diseases and in healthy subjects. The inventors postulated that genes relating to early stage allergic disease could be found by selecting genes whose expression levels in eosinophils differ between patients with early stage allergic diseases and healthy subjects by comparing expression levels in patients at different stages of advancement of allergic diseases with those in healthy subjects.

As a result of analyzing the expression levels of genes in the peripheral blood eosinophils based on such strategy, the present inventors confirmed that the following 17 genes all showed significantly increased expression in the eosinophils of patients with early stage allergic diseases. These genes respectively comprise the nucleotide sequences of the following SEQ ID NOS.

“1858-05”/SEQ ID NO: 1

“1901-21”/SEQ ID NO: 2

“1913-17”/SEQ ID NO: 3

“1852-09”/SEQ ID NO: 4

“1945-03”/SEQ ID NO: 5

“1948-16”/SEQ ID NO: 6

“1833-02”/SEQ ID NO: 7

“1873-30”/SEQ ID NO: 8

“1937-03”/SEQ ID NO: 9

“1949-02”/SEQ ID NO: 10

“1956-04”/SEQ ID NO: 11

“1919-13”/SEQ ID NO: 12

“1917-03”/SEQ ID NO: 13

“1941-20”/SEQ ID NO: 14

“1930-03”/SEQ ID NO: 15

“1921-05”/SEQ ID NO: 16

“1925-08”/SEQ ID NO: 17

Hereinafter, in the present description, these genes are individually referred to as the “1858-05” gene, “1901-21” gene, “1913-17” gene, “1852-09” gene, “1945-03” gene, “1948-16” gene, “1833-02” gene, “1873-30” gene, “1937-03” gene, “1949-02” gene, “1956-04” gene, “1919-13” gene, “1917-03” gene, “1941-20” gene, “1930-03” gene, “1921-05” gene, and “1925-08“gene. Furthermore, the proteins encoded by these genes are individually referred to as the “1858-05” protein, “1901-21” protein, “1913-17” protein, “1852-09” protein, “1945-03” protein, “1948-16” protein, “1833-02” protein, “1873-30” protein, “1937-03” protein, “1949-02” protein, “1956-04” protein, “1919-13” protein, “1917-03” protein, “1941-20” protein, “1930-03” protein, “1921-05” protein, and “1925-08” protein. A database search for these genes found genes containing nucleotide sequences having homology to the following genes:

“1833-02” gene: unknown function (KIAA0006, Accession No. D13631)

“1873-30” gene: nucleotide sequence NM_{—}017719.1;

“1949-02” gene: nucleotide sequence HSA23852;

“1917-03” gene: nucleotide sequence that is considered to encode a secretory protein (KIAA1245; GenBank AB033071); and

“1925-08” gene: similarly a nucleotide sequence that is considered to encode a secretory protein (X97610; GenBank Z09912).

None of these genes have been suggested to be related to allergic diseases.

The other genes were considered novel since their nucleotide sequences could not be found in known genetic databases.

Finally, the present inventors discovered that testing of an allergic disease, and screening of candidate compounds for a therapeutic agent for an allergic disease could be performed using the expression level of these genes as an indicator, and thereby completed this invention.

Specifically, this invention relates to genes showing high levels of expression in the early stage allergic disease and uses thereof. More specifically, this invention relates to a method of testing for an allergic disease using expression of the gene as an indicator, a method of detecting an influence of candidate compounds on the expression of such a gene, and in addition, a method of screening of candidate compounds for a therapeutic agent for an allergic disease based on this detection method.

[1] A method of testing for an early stage allergic disease, said method comprising the steps of:

a) measuring the expression level of a gene comprising the nucleotide sequence selected from the group consisting of SEQ ID NOs: 1, 2, 3, 4, 5, 6, 7, 8, 9, 10, 11, 12, 13, 14, 15, 16, and 17 in eosinophil cells of a test subject; and

b) comparing the measured expression level to the expression level of the same gene in eosinophil cells of a healthy subject.

[2] The testing method of [1], wherein the allergic disease is atopic dermatitis.

[3] The testing method of [1], wherein the expression level of a gene is measured by cDNA PCR.

[4] A reagent for testing for the presence of an early stage allergic disease, said reagent comprising an oligonucleotide that is at least 15 nucleotides long and comprises a nucleotide sequence complementary to a polynucleotide having the nucleotide sequence selected from the group consisting of SEQ ID NOs: 1, 2, 3, 4, 5, 6, 7, 8, 9, 10, 11, 12, 13, 14, 15, 16, and 17 or to its complementary strand.

[5] A method of detecting an influence of a candidate compound on the expression level of a polynucleotide of (a) or (b), said method comprising the steps of:

(1) contacting the candidate compound with a cell that expresses a polynucleotide of (a) or (b):

(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;

(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17; and

(2) measuring the expression level of the polynucleotide of (a) or (b) of (1).

[6] The method of [5], wherein the cell is derived from a leukocyte cell line.

[7] A method of detecting an influence of a candidate compound on the expression level of a polynucleotide of (a) or (b):

(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;

(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17;

said method comprising the steps of:

(1) administering the candidate compound to a test animal; and

(2) measuring the expression intensity of the polynucleotide of (a) or (b) in the eosinophil cells of the test animal.

[8] A method of screening for a compound that decreases the expression level of the polynucleotide of (a) or (b) above, the method comprising the steps of detecting an influence on the expression level by the method of [5] or [7], and selecting a compound that decreases the expression level compared to a control.

[9] A method of detecting an influence of a candidate compound on the activity of a transcription regulatory region of a gene comprising the nucleotide sequence selected from the group consisting of SEQ ID NOs: 1, 2, 3, 4, 5, 6, 7, 8, 9, 10, 11, 12, 13, 14, 15, 16, and 17, said method comprising the steps of:

(1) contacting a candidate compound with a cell transfected with a vector comprising the transcription regulatory region of the gene containing the nucleotide sequence selected from the group consisting of SEQ ID NOs: 1, 2, 3, 4, 5, 6, 7, 8, 9, 10, 11, 12, 13, 14, 15, 16, and 17, and a reporter gene that is expressed under the control of the transcription regulatory region; and

(2) measuring the activity of the reporter gene.

[10] A method of screening for a compound that decreases the activity of the transcription regulatory region of a gene containing the nucleotide sequence selected from the group consisting of SEQ ID NOs: 1, 2, 3, 4, 5, 6, 7, 8, 9, 10, 11, 12, 13, 14, 15, 16, and 17, said method comprising the steps of detecting an influence of a candidate compound on the activity by the method of [9], and selecting a compound that decreases the activity compared to a control.

[11] A vector comprising the transcription regulatory region of a gene containing the nucleotide sequence selected from the group consisting of SEQ ID NOs: 1, 2, 3, 4, 5, 6, 7, 8, 9, 10, 11, 12, 13, 14, 15, 16, and 17, and a reporter gene that is expressed under the control of the transcription regulatory region.

[12] A cell carrying the vector of [11].

[13] A therapeutic agent for an allergic disease, said agent comprising as the active ingredient, a compound obtainable by the method of screening of [8] or [10].

[14] A therapeutic agent for an allergic disease, which comprises, as a principal ingredient, an antisense DNA against a polynucleotide having the nucleotide sequence selected from the group consisting of SEQ ID NOs: 1, 2, 3, 4, 5, 6, 7, 8, 9, 10, 11, 12, 13, 14, 15, 16, and 17, or a portion thereof.

[15] A therapeutic agent for an allergic disease, which comprises, as a principal ingredient, an antibody against a peptide consisting of an amino acid sequence of “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” protein.

[16] A polynucleotide of (a) or (b):

(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;

(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17.

[17] A protein encoded by the polynucleotide of [16].

[18] A vector that harbors the polynucleotide of [16] in an expressible state.

[19] A transformed cell that harbors the polynucleotide of [16], or the vector of [18].

[20] A method of producing the protein of [17], said method comprising the steps of culturing the transformed cell of [19], and collecting its expression product.

[21] An antibody against the protein of [17].

[22] A method of immunologically measuring the protein of [17], said method comprising the step of observing the immunological reaction between the antibody of [21] and the protein of [17].

[23] An oligonucleotide having at least 15 nucleotides long, and comprising a nucleotide sequence complementary to a polynucleotide having the nucleotide sequence selected from the group consisting of SEQ ID NOs: 1, 2, 3, 4, 5, 6, 7, 8, 9, 10, 11, 12, 13, 14, 15, 16, and 17, or to its complementary strand.

[24] A method of measuring the polynucleotide of [16], said method comprising the step of observing hybridization of the oligonucleotide of [23] to the polynucleotide of [16].

[25] An early stage allergic disease model animal, wherein said animal is a transgenic non-human vertebrate, in which expression intensity of the polynucleotide of (a) or (b) in eosinophil cells is increased:

(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;

(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17.

[26] A kit for screening for a candidate compound for a therapeutic agent for an allergic disease, said kit comprising cells that express a gene comprising the nucleotide sequence selected from the group consisting of SEQ ID NOs: 1, 2, 3, 4, 5, 6, 7, 8, 9, 10, 11, 12, 13, 14, 15, 16, and 17, and a polynucleotide that is at least 15 nucleotides long and hybridizes to the nucleotide sequence selected from the group consisting of SEQ ID NOs: 1, 2, 3, 4, 5, 6, 7, 8, 9, 10, 11, 12, 13, 14, 15, 16, and 17 or to its complementary strand.

[27] A kit for screening for a candidate compound for a therapeutic agent for an allergic disease, said kit comprising cells that express a gene comprising the nucleotide sequence selected from the group consisting of SEQ ID NOs: 1, 2, 3, 4, 5, 6, 7, 8, 9, 10, 11, 12, 13, 14, 15, 16, and 17, and an antibody that recognizes the peptide comprising the amino acid sequence of “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” protein.

The present invention relates to a method of testing for the presence of an early stage allergic disease using novel genes associated with allergic diseases, using the expression level of these genes in eosinophil cells as indicators of disease. Hereinafter, these 17 genes are generally referred as genes of this invention. Each of the genes of this invention comprises the nucleotide sequence of SEQ ID NOs: 1 to 17, respectively.

The nucleotide sequences shown in SEQ ID NOs: 1 to 17 are partial sequences of the full length cDNAs, respectively. The full length cDNA containing this partial sequence can be obtained by screening a cDNA library of leukocytes with a probe comprising a nucleotide sequence selected from the nucleotide sequences of SEQ ID NOs: 1 to 17. The genes of this invention are expressed in eosinophils. Therefore, either a cDNA library derived from eosinophil cells, or from a group of cells comprising eosinophils can be used to obtain the genes of this invention. Furthermore, the genes of this invention include those of which expressions were confirmed in cells other than eosinophils. These genes can be obtained by using a cDNA library derived from cells other than eosinophils. For example, expression of 1913-17, 1956-04, 1921-05, and such have been observed in neutrophils as well. Furthermore, some genes, including 1873-30 and 1917-03, were found to be expressed in a wide variety of cells besides eosinophils, including neutrophils, B-cells, T-cells, and monocytes.

Furthermore, the sequences of the genes of this invention can be extended by the RACE method (Frohman, M. A. et al.: Proc. Natl. Acad. Sci. USA, 85: 8992, 1988). Specifically, extended cDNA can be obtained by using the sequence derived from the genes of this invention as a primer, converting the mRNA of leukocytes and such into single stranded cDNA, adding an oligomer to its terminal end, then performing PCR.

Full length cDNAs of the genes of this invention, which may be isolated in this manner based on the sequence information of the cDNAs of SEQ ID NOs: 1 to 17 of this invention, are included in “polynucleotides comprising the nucleotide sequences of SEQ ID NOs: 1 to 17” of this invention. Furthermore, based on the nucleotide sequences of cDNAs obtained in this manner, the amino acid sequences encoded by the cDNAs can be determined.

The present invention relates to a polynucleotide comprising the nucleotide sequence of any one of SEQ ID NOs: 1 to 17. This invention also relates to a polynucleotide that hybridizes under stringent conditions to a polynucleotide comprising the nucleotide sequence of any one of SEQ ID NOs: 1 to 17 and that encodes a protein functionally equivalent to the protein encoded by the polynucleotide comprising the nucleotide sequence of any one of SEQ ID NOs: 1 to 17. In this invention, the term “polynucleotide” includes a natural nucleic acid molecule such as DNA and RNA, and artificial molecules comprising labeled molecule and various nucleotide derivatives. Artificial polynucleotides include polynucleotides having the phosphorothioate bond and peptide bond as a backbone.

These polynucleotides according to this invention can be chemically synthesized, or isolated from natural nucleic acids such as mRNA, a cDNA library, or a genomic library. Polynucleotide molecules according to this invention are useful for the production of protein encoded by them, inhibiting the expression of the genes of this invention as antisense nucleic acids, or as the probes for detecting their presence by hybridization.

Furthermore, in this invention, when expression of a certain protein increases in eosinophils of a patient or an animal with early stage allergic disease, this protein is said to be functionally equivalent to the protein of this invention. The increase in expression of a certain protein in eosinophils can be confirmed by comparing the expression levels of the gene encoding this protein in collected eosinophils.

A polynucleotide that hybridizes under stringent conditions to the polynucleotide comprising the nucleotide sequence of any one of SEQ ID NOs: 1 to 17 and that encodes a functionally equivalent protein can be obtained by known techniques such as hybridization and PCR based on the nucleotide sequence of any one of SEQ ID NOs: 1 to 17. For example, cDNA comprising a nucleotide sequence that is highly homologous to that of any one of SEQ ID NOs: 1 to 17 can be obtained by screening a leukocyte cDNA library using an oligonucleotide comprising any one of nucleotide sequences selected from the nucleotide sequences of SEQ ID NOs: 1 to 17 as a probe under stringent conditions. When a polynucleotide hybridizes to the polynucleotide comprising the nucleotide sequence of any one of SEQ ID NOs: 1 to 17 under stringent conditions, in most cases, such a protein encoded by the polynucleotide is thought to have the activity similar to that of the protein of this invention. Stringent conditions mean hybridization in 4×SSC at 65° C. followed by washing with 0.1×SSC at 65° C. for 1 hour. Temperature conditions for hybridization and washing that greatly influence stringency can be adjusted according to the melting temperature (Tm). Tm varies with the ratio of constitutive nucleotides in the hybridizing base pairs, and the composition of hybridization solution (concentrations of salts, formamide, and sodium dodecyl sulfate). Therefore, considering these conditions, those skilled in the art can select an appropriate condition to produce an equal stringency from their experience.

A protein encoded by cDNA comprising the nucleotide sequence that has a high identity to the cDNA of this invention would be a functionally equivalent protein in this invention. Herein, a nucleotide sequence with a high identity refers to a nucleotide sequence that shows 70% or more homology in general, usually 80% or more, preferably 90% or more, more preferably 95% or more, furthermore preferably 98% or more, and specifically preferably 99% or more identity with a nucleotide sequence of this invention. The degree of identity of one nucleotide sequence to another can be determined by following the well-known algorism such as BLASTN.

Alternatively, a gene encoding a protein having, for example, 90% or more, preferably 95% or more, and furthermore preferably 99% or more homology to the amino acid sequence of “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” protein can be referred to as a gene functionally equivalent to the “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” gene, respectively.

A cDNA with a high identity to a cDNA of this invention can be obtained by PCR performed using oligonucleotides comprising the nucleotide sequence of any one of SEQ ID NOs: 1 to 17 as the primers and a leukocyte cDNA library as a template. If human cells are used as a source of cDNA, it is possible to obtain human cDNA. When cells from vertebrates other than humans are used, it is possible to obtain the counterpart of human CDNA in different animal species. Examples of such non-human animals include various experimental animals such as mice, rats, dogs, pigs, and goats. Counterparts of the genes of this invention in experimental animals are useful in preparing allergic disease animal models from various animal species and as the marker in developing therapeutic agents for allergic diseases.

A gene that can be amplified using, as primers, oligonucleotides comprising nucleotide sequences selected from the nucleotide sequences of SEQ ID NOs: 1 to 17 used in Examples and that encodes a protein whose expression significantly increases in eosinophils of patients with early stage allergic diseases is also a functionally equivalent gene. In this invention, the genes comprising the nucleotide sequences of any one of SEQ ID NOs: 1 to 17or a gene functionally equivalent thereto is referred to as an indicator gene. A protein encoded by an indicator gene is termed an indicator protein.

This invention also relates to oligonucleotides comprising nucleotide sequences complementary to the polynucleotides having the nucleotide sequence of any one of SEQ ID NOs: 1 to 17 or to a complementary strand thereof, and that are at least 15-nucleotide-long. Herein, the term “complementary strand” is defined as one strand of a double stranded polynucleotide composed of A:T (U for RNA) and G:C base pairs to the other strand. In the context of the present invention, “complementary” strands need not be completely homologous within a region of at least 15 continuous nucleotides, provided they have at least 70%, preferably at least 80%, more preferably 90%, and even more preferably 95% or higher homology within that region. The degree of homology of one nucleotide sequence to another can be determined by known algorithms, such as BLAST.

An oligonucleotide of the present invention is useful for detecting and synthesizing a polynucleotide of this invention. Techniques for detecting or synthesizing the target nucleotides using oligonucleotides as the probe or primer are known. For example, Northern blot technique with mRNA as a target polynucleotide is a typical method of detecting RNA. RT-PCR that uses mRNA as a template enables the synthesis of the polynucleotide of this invention. Furthermore, it is also possible to find out the presence of mRNA as well as its expression level using the presence and amount of that synthetic product as an indicator. Alternatively, a polynucleotide of this invention that is expressed in eosinophils can be detected by an in situ hybridization technique.

Furthermore, using a polynucleotide of this invention, a protein encoded thereby can be produced as a recombinant. More specifically, a transformant is obtained by inserting the coding region of the polynucleotide having the nucleotide sequence of any one of SEQ ID NO: 1 to 17 into a known expression vector, and transfecting an appropriate host with the resulting recombinant vector. Alternatively, a transformant may be obtained by integrating the polynucleotide containing the coding region into a genome of an appropriate host.

A protein of this invention can be obtained by culturing the resulting transformant under the conditions in which a polynucleotide of this invention can be expressed and collecting the expression product. The expression product can be purified by known techniques.

In addition, the present invention also relates to a protein encoded by a polynucleotide of this invention. A protein of this invention is useful as an indicator for diagnosing an allergic disease, such as atopic dermatitis.

Additionally, a protein of the present invention and its fragments are useful as an antigen for producing an antibody against the selected protein. Techniques for obtaining an antibody using a given antigen are well known in the art. That is, a protein or its fragment is mixed with an appropriate adjuvant, and the antigen thus prepared is inoculated to an animal to be immunized. There is no limitation in the type of animals to be immunized. Typical examples of animals to be immunized include mice, rats, rabbits, and goats. After the increase in the antibody titer is confirmed, blood is collected, and the serum is fractionated as an antiserum. The IgG fraction may be further purified to obtain a purified antibody. For the purification of antibody, techniques such as ammonium sulfate precipitation, ion exchange chromatography, immunoaffinity chromatography using protein A-conjugated Sepharose and the protein of this invention as the ligand can be utilized.

Furthermore, it is also possible to obtain a monoclonal antibody by transforming an antibody-producing cell using techniques such as cell fusion, and cloning the resulting transformant. Alternatively, a method of isolating a gene of the antibody-producing cell and constructing a humanized antibody and chimeric antibody is also known. The antibody thus obtained is useful as a tool for immunologically measuring the protein of this invention. A protein of the present invention can be immunologically assayed by contacting the protein with the antibody, and observing an immunological reaction between the two. Various known assay formats can be applied to the immunoassay according to this invention. For example, a protein contained in a sample such as serum can be measured by ELISA or such. Antibody-based detection of a protein expressed in eosinophils can be performed using immunohistochemical technique or fluorescence activated cell sorter (FACS) using a fluorescence labeled antibody.

Herein, the term “allergic disease” is a general term for diseases in which allergic reaction is involved. More specifically, it is defined as a disease in which an allergen must be identified, a strong correlation between the exposure to the allergen and the onset of the pathological change must be demonstrated, and the pathological change must be proven to have an immunological mechanism. Herein, an immunological mechanism means that immune responses by the leukocytes are induced by the stimulation of the allergen. Examples of known allergens include mite antigen, and pollen antigen.

Representative allergic diseases include bronchial asthma, allergic rhinitis, atopic dermatitis, pollen allergy, and insect allergy. Allergic diathesis is a genetic factor that is inherited from allergic parents to their children. Familial allergic diseases are also called atopic diseases, and the causative factor that is inherited is the atopic diathesis. The term “atopic dermatitis” is a general term for atopic diseases with dermatitis among atopic diseases.

All of the genes of this invention showed increased expression levels in the eosinophils of patients with light atopic dermatitis compared to those of healthy subjects. Therefore, allergic diseases can be tested using the expression levels of the genes of this invention as indicators. In the test method of this invention, the expression level of any one or a plurality of genes selected from the 17 genes of this invention is used as the indicator. In the test method of this invention, not only the genes of this invention, but other indicators for allergic disease can be used in combination. The testing based on a plurality of indicators allows a more accurate determination.

For example, the testing method for diagnosing an allergic disease of this invention includes the following methods. Specifically, an increase in the expression level of one or more genes of this invention in a patient showing early symptoms suspect of an allergic disease, proves that the early symptoms in that patient is caused by an allergic disease.

Herein, the expression levels of the genes of this invention include the transcription of the genes to mRNAs as well as the translation into proteins. Therefore, a method for testing for allergic disease according to the present invention may be performed by comparing either the expression intensity of mRNAs corresponding to the genes, or the expression levels of a proteins encoded by the genes.

Measurement of the expression levels of the genes of this invention in a test for allergic diseases of the present invention may be conducted according to known gene analytical methods. More specifically, for example, a hybridization technique with nucleic acids that hybridize to the genes as a probe, a gene amplification technique with DNAs hybridizing to the genes of this invention as a primer, or such can be utilized.

A polynucleotide that has at least 15 nucleotides and that is complementary to a polynucleotide comprising the nucleotide sequence of any one of SEQ ID NOs: 1 to 17 or the complementary strand thereof can be used as a primer or probe for the test according to the present invention. Herein, the term “complementary strand” means one strand of a double stranded DNA composed of A:T (U for RNA) and G:C base pairs to the other strand. In addition, “complementary” encompasses both those nucleotides completely complementary to a region of at least 15 continuous nucleotides, as well as those having a homology of at least 70%, preferably at least 80%, more preferably 90%, and even more preferably 95% or higher. The degree of homology between nucleotide sequences can be determined by the algorithm, such as BLAST.

Such polynucleotides can be useful as the probe to detect and isolate the polynucleotide encoding the protein according to the present invention, or as the primer to amplify the polynucleotide according to the present invention. When used as a primer, those polynucleotides comprise usually 15 bp to 100 bp, preferably 15 bp to 35 bp of nucleotides. When used as a probe, DNAs comprising the whole sequence of a polynucleotide according to the present invention, or a partial sequence thereof that contains at least 15-bp nucleotides maybe used. When used as a primer, the 3′ region thereof must be complementary to the indicator gene, while the 5′ region can be linked to a restriction enzyme-recognition sequence or tag.

The “polynucleotides” of the present invention may be either DNA or RNA. These polynucleotides may be either synthetic or naturally-occurring. Also, DNAs used as probes for hybridization are usually labeled. Examples of labeling methods include those as described below. Herein, the term “oligonucleotide” means a polynucleotide with relatively low degree of polymerization. Oligonucleotides are included in polynucleotides.

nick translation labeling using DNA polymerase I;

end labeling using polynucleotide kinase;

fill-in end labeling using Klenow fragment (Berger, S L, Kimmel, A R. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. (1984) Nucleic Acid Res., 12, 7035-7056); and

non-isotopic labeling of DNA by incorporating modified nucleotides (Kricka, L J. (1992) Nonisotopic DNA Probing Techniques. Academic Press).

For testing for the presence of an allergic disease using hybridization techniques, for example, Northern hybridization, dot blot hybridization, or DNA microarray technique may be used. Furthermore, gene amplification techniques, such as RT-PCR method, may be used. By using the PCR amplification monitoring method during the gene amplification step in RT-PCR, one can achieve more quantitative analysis for the gene expression of the present invention.

In the PCR gene amplification monitoring method, the detection target (DNA or reverse transcript of RNA) is hybridized to probes that are dual-labeled at both ends with different fluorescent dyes whose fluorescences cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al. 1991, Proc. Natl. Acad. Sci. USA 88: 7276-7280; Livak, K. J. et al., 1995, PCR Methods and Applications 4(6): 357-362; Heid, C. A. et al., 1996, Genome Research 6: 986-994; Gibson, E. M. U. et al., 1996, Genome Research 6: 995-1001). For the PCR amplification monitoring method, for example, ABI PRISM7700 (PE Biosystems) may be used.

The method of testing for allergic diseases of the present invention can also be carried out by detecting a protein encoded by one or more genes of this invention. Such test methods include, for example, those utilizing antibodies binding to a protein encoded by such a gene, including the Western blotting method, the immunoprecipitation method, and the ELISA method.

Antibodies that bind to the proteins encoded by the genes of this invention used in the detection may be produced by techniques known to those skilled in the art. Antibodies used in the present invention may be polyclonal or monoclonal antibodies (Milstein, C. et al., 1983, Nature 305 (5934): 537-40). For example, polyclonal antibodies against the proteins encoded by the genes of the present invention may be produced by collecting blood from mammals sensitized with an antigen, and separating the serum from this blood using known methods. As polyclonal antibodies, the serum containing polyclonal antibodies may be used. According to needs, a fraction containing polyclonal antibodies can be further isolated from this serum. Alternatively, a monoclonal antibody can be obtained by isolating immune cells from mammals sensitized with an antigen; fusing these cells with myeloma cells, and such; cloning hybridomas thus obtained; and collecting the antibody from the culture as the monoclonal antibody.

To detect the proteins encoded by the genes of this invention, these antibodies may be appropriately labeled. Alternatively, instead of labeling the antibodies, a substance that specifically binds to antibodies, for example, protein A or protein G, may be labeled to arrange an indirect detection of the proteins. More specifically, one example of an indirect detection method is ELISA.

A protein or partial peptides thereof that is used as an antigen may be obtained, for example, by inserting the genes or portion thereof into an expression vector, introducing it into an appropriate host cell to produce a transformant, culturing the transformant to express the recombinant protein, and purifying the expressed recombinant protein from the culture or the culture supernatant. Alternatively, oligonucleotides consisting of the amino acid sequence encoded by the gene, or partial amino acid sequences of the amino acid sequence encoded by the full-length cDNA obtained based on SEQ ID NOs: 1 to 17 are chemically synthesized to be used as the antigen.

In this invention, eosinophil cells of a test subject are used as the sample. Eosinophil cells can be prepared by conventional methods from the peripheral blood. Specifically, leukocytes can be isolated, for example, by fractionating heparinized blood by centrifugation. Next, granulocytes can be fractionated, for example, by Ficoll centrifugation of leukocytes, and furthermore eosinophil cells can be isolated, for example, by depletion of neutrophils using the CD16 antibody. A sample for immunological assays of the aforementioned protein can be obtained by disrupting the isolated eosinophils to produce a lysate. Alternatively, a sample for measuring mRNA corresponding to the aforementioned gene can be obtained by extracting mRNA from this lysate. The use of a commercially available kit is convenient for extracting mRNA and preparing a lysate of eosinophils.

Alternatively, the expression level of a gene that serves as an indicator in this invention may be measured not in isolated in eosinophils, but in the whole blood, and peripheral blood leukocyte population. In this case, by correcting the measured values, the change of gene expression levels in cells can be determined. For example, based on the measured value of the expression level of a gene (housekeeping gene), whose expression level is eosinophil specific and is not widely altered regardless of the cellular conditions, the measured value of the expression level of the gene serving as an indicator in this invention can be corrected.

Alternatively, in the case where the protein to be detected is a secretory protein, comparison of the expression level of a gene encoding the protein is accomplished by measuring the amount of the target protein contained in body fluid sample, such as blood and serum, in a subject.

In the method of testing for an allergic disease of this invention, these genes that show increased expression in light allergic diseases are used as indicators for early symptoms of an allergic disease.

Furthermore, the present invention relates to an animal model for an allergic disease, wherein said animal is a transgenic non-human animal in which the expression level of the polynucleotide of the following (a) or (b) is increased in eosinophil cells:

(a) polynucleotides comprising the nucleotide sequences of SEQ ID NO: 1 to SEQ ID NO: 17; and

(b) polynucleotides that hybridize under stringent conditions to the DNAs comprising the nucleotide sequences of SEQ ID NO: 1 to SEQ ID NO: 17 and encode proteins that show increased expression in eosinophils of a patient with an early stage allergic disease.

According to this invention, the expression levels of the aforementioned indicator genes in eosinophil cells were found to increase in eosinophils of patients with early stage atopic dermatitis. Therefore, animals in which the expression level of these genes or genes that are functionally equivalent thereto in eosinophil cells are artificially enhanced can be utilized as animal models for early stage allergic diseases. The phrase “increase of the expression level of indicator genes in eosinophils” includes increase of their expression level in the entire population of leukocytes. That is, the expression level of the aforementioned genes may be increased not only in eosinophils alone, but also in the entire population of leukocytes. The functionally equivalent genes of this invention refers to genes encoding proteins having activities similar to known activities of proteins encoded by each of the indicator genes. A representative example of a functionally equivalent gene is a counterpart of an indicator gene originally present in the animal species of the transgenic animal.

The genes showing increased expression in early stage allergic diseases can be said to be genes that regulate, at the upstream position, the pathology of allergic diseases. In other words, pathology of allergies is considered to appear when genes that act in early stage allergic diseases regulate expression or suppression of various genes positioned downstream therefrom. Thus, genes that show increased expression in early stage allergic diseases can be considered to be genes that play an important role in pathologic formation of allergies. Therefore, in allergy therapy, pharmaceutical agents that suppress the expression or inhibit the activity of these genes can be expected to have the function of not only simply improving allergic symptoms, but also eliminating the fundamental cause of pathologic formation of allergies.

As described above, a gene the expression level of which is increased in early stage allergic disease is very important. Therefore, it is highly significant to assess the role of the gene and the effects of drugs targeting this gene using transgenic animals, which can be obtained by elevating the expression level of this gene in vivo, as the early stage allergic disease model animal.

Early stage allergic disease model animals according to the present invention are useful in not only in screening drugs for treating or preventing early stage allergic diseases as described below but also in elucidating mechanisms of early stage allergic diseases, furthermore, testing the safety of compounds screened.

For example, if early stage allergic disease animal models according to the present invention either develop clinical manifestations of dermatitis or show changes in measured values related to any allergic disease, it is possible to construct a screening system to find a compound having activity to recover normal conditions.

In the present invention, an increase in the expression level means the state wherein a target gene is transduced as a foreign gene and forcibly expressed; the state wherein transcription of a gene inherent in the host and translation thereof into protein are increased; or the state wherein decomposition of the translation product, protein, is suppressed. Gene expression levels can be confirmed by, for example, the quantitative PCR as described in Examples. Furthermore, the activity of a translation product, protein, can be confirmed by comparing to that occurring in a normal state.

A typical transgenic animal is the one to which a gene of interest is transduced to be forcibly expressed. Examples of other types of transgenic animals are those in which a mutation is introduced into the coding region of the gene to increase its activity or to modify the amino acid sequence of the gene product protein so as to be hardly decomposed. Examples of mutations in the amino acid sequence include the substitution, deletion, insertion, or addition of amino acid(s). In addition, by mutagenizing the transcriptional regulatory region of the gene, the expression itself of the gene of this invention can be controlled.

Methods for obtaining transgenic animals with a particular gene as a target are well known in the art. That is, a transgenic animal can be obtained by a method wherein the gene and ovum are mixed and treated with calcium phosphate; a method where the gene is introduced directly into the nucleus of oocyte in pronuclei with a micropipette under a phase contrast microscope (microinjection method, U.S. Pat. No. 4,873,191); or a method wherein embryonic stem cells (ES cells) are used. Furthermore, there have been developed methods for infecting ovum with a gene-inserted retrovirus vector, a sperm vector method for transducing a gene into ovum via sperm, and such. The sperm vector method is a gene recombination technique for introducing a foreign gene by fertilizing ovum with sperm after a foreign gene has been incorporated into sperm by the adhesion or electroporation method, and so on (M. Lavitranoet, et al. Cell, 57, 717, 1989).

Transgenic animals used as animal models for early stage allergic diseases of the present invention can be produced using all the vertebrates except for humans. More specifically, transgenic animals having various transgenes and having modified gene expression levels thereof can be produced using vertebrates such as mice, rats, rabbits, miniature pigs, goats, sheep, or cattle.

Furthermore, this invention relates to a method of detecting an influence of a candidate compound on the expression level of a polynucleotide of this invention. In this invention, the genes of this invention show significant increase in expression in the eosinophils of patients with light atopic dermatitis. Therefore, based on the method of detecting an influence on the expression level of the gene, a therapeutic agent for an allergic disease can be obtained by selecting a compound that can decrease the expression level of the gene. In the present invention, a compound that decreases the expression level of the gene is a compound having the effect of inhibiting any one of the steps of transcription of the gene, translation, and expression of protein activity.

The method of detecting an influence of a candidate compound on the expression level of a polynucleotide of this invention can be performed in vivo or in vitro. In order to detect an influence in vivo, an appropriate test animal is used. For example, animal models for allergic diseases and those comprising non-human transgenic animals in which expression of a gene of above-described (a) or (b) is increased in eosinophil cells can be used as the test animal. Detection of an influence on the expression level in vivo based on the present invention can be performed, for example, by the steps of:

(1) administering a candidate compound to a test animal; and

(2) measuring the expression level of a polynucleotide of aforementioned (a) or (b) in eosinophil cells of the test animal.

An influence of a candidate compound for a pharmaceutical agent on the expression level of the genes of this invention can be detected by administering the candidate compound to the model animal, in which the expression levels of one or more genes are increased, and monitoring the effect of the compound towards expression of the genes of this invention in eosinophils of the model animal. Furthermore, a candidate compound can be screened by selecting the candidate compound that decreases the expression level of one or more genes of this invention based on the detection results.

Such screening allows for the selection of drugs that are involved in various ways in the expression of the genes of this invention. Specifically, for example, a candidate compound for a pharmaceutical agent having the following action can be discovered:

Suppression of a signal transduction pathway that causes expression of one or more genes of this invention;

Suppression of transcription activity of one or more genes of this invention; and

Facilitation of degradation of the transcription product of one or more genes of this invention.

An in vitro detection can be performed, for example, by a method wherein a candidate compound is contacted with cells expressing a gene according to above-descried (a) or (b) to detect expression levels of these genes. More specifically, the method may be carried out according to the following steps of:

(1) contacting a candidate compound with cells that express a polynucleotide according to above-described (a) or (b); and

(2) measuring the expression level of the polynucleotide according to above-described (a) or (b).

In this invention, cells to be used in the step (1) can be obtained by inserting these polynucleotides into an appropriate expression vector and then transfecting suitable host cells with the vector. Any vectors and host cells may be used, so long as they are capable of expressing the gene of this invention. Examples of host cells in the host-vector system are Escherichia coli cells, yeast cells, insect cells, animal cells, and available vectors usable for each can be selected.

Vectors may be transfected into the host by biological methods, physical methods, chemical methods, and so on. Examples of biological methods include methods using virus vectors; methods using specific receptors; the cell-fusion method (HVJ (Sendai virus) method; the polyethylene glycol (PEG) method; the electric cell fusion method, and microcell fusion method (chromosome transfer)). Examples of physical methods include the microinjection method, the electroporation method, and the method using gene particle gun. The chemical methods are exemplified by the calcium phosphate precipitation method, the liposome method, the DEAE-dextran method, the protoplast method, the erythrocyte ghost method, the erythrocyte membrane ghost method, and the microcapsule method.

In the detection method of this invention, leukocyte cell lines can be used as cells for expressing a polynucleotide of the aforementioned (a) or (b). Examples of leukocyte cell lines are cell lines derived from leukocytes, such as Eol, YY-1, HL-60, TF-1, and AML14.3D10. Among the leukocyte cell lines, cell lines derived from eosinophils are preferred for the detection method of this invention. The following are cell lines derived from eosinophils:

Eol;

YY-1; and

AML14.3D10.

Eol (Eol-1: Saito H et al., Establishment and characterization of a new human eosinophilic leukemia cell-line. Blood 66, 1233-1240, 1985) can be obtained from Hayashibara Research Institute. Similarly, YY-1 (Ogata N et al., The activation of the JAK2/STAT5 pathway is commonly involved in signaling through the human IL-5 receptor. Int. Arch. Allergy Immunol., Suppl 1, 24-27, 1997) is available from The Institute of Cytosignal Research. Furthermore, AML14. 3D10 (Baumann MA et al., The AML14 and AML14.3D10 cell lines: a long-overdue model for the study of eosinophils and more. Stem Cells, 16, 16-24, 1998) is commercially available from Paul CC at Research Service, VA Medical Center, Dayton, Ohio, USA.

In addition, by culturing in the presence of butyric acid for about 1 week, HL-60 clone 15 (ATCC CRL-1964), which is an undifferentiated leukocyte cell line, can differentiate into eosinophils to give an eosinophil cell line. Eosinophils can be detected due to their morphological characteristic of being polymorphonuclear and having eosinophilic granules. Morphological observations are performed by Giemsa staining and Difquick staining. Generally, human leukocyte cell lines including eosinophils can be established by cloning immortalized cells from a leukemia patient sample. Therefore, those skilled in the art can obtain eosinophil cell lines by a conventional method when necessary.

The method of screening first involves contacting a candidate compound with the aforementioned leukocyte cell line. Then, the expression levels of one or more polynucleotides of (a) or (b) in the leukocyte cell line are measured and a compound that decreases the expression level of one or more genes is selected.

In the method of the present invention, expression levels of polynucleotides according to above-described (a) or (b) can be compared by detecting the expression levels of not only proteins encoded by these genes but also the corresponding mRNAs. For the comparison of the expression level using mRNA, the step of preparing mRNA sample as described above is conducted in place of the step of preparing a protein sample. Detection of mRNA and protein can be carried out according to the known methods as described above.

Furthermore, based on the disclosure of this invention, it is possible to obtain the transcriptional regulatory region of the gene of the present invention and to construct a reporter assay system. In the context of the present invention, a reporter assay system refers to an assay system for screening for a transcriptional regulatory factor that acts on the transcriptional regulatory region by using the expression level of a reporter gene that is located downstream of the transcriptional regulatory region and expressed under the control of the regulatory region as an indicator.

More specifically, this invention relates to a method of screening for therapeutic agents for an allergic disease, the method comprising the steps of:

(1) contacting a candidate compound with a cell transfected with a vector containing the transcription regulatory region of an indicator gene and a reporter gene that is expressed under the control of this transcription regulatory region;

(2) measuring the activity of the reporter gene; and

(3) selecting a compound that decreases the expression level of the reporter gene compared to a control,

wherein the indicator gene is a gene selected from the group consisting of “1858-05”, “1901-21”, “1913-17-, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, and “1925-08” and genes functionally equivalent thereto.

A transcriptional regulatory region is exemplified by promoter, enhancer, as well as CAAT box, and TATA box, which are usually found in the promoter region. Examples of reporter genes include the chloramphenicol acetyltransferase (CAT) gene, the luciferase gene, and growth hormone genes.

A transcriptional regulatory region of a gene of the present invention can be obtained as follows. Specifically, first, based on the nucleotide sequence of a cDNA disclosed in this invention, a human genomic DNA library, such as BAC library and YAC library, is screened by a method using PCR or hybridization to obtain a genomic DNA clone containing the sequence of the cDNA. Based on the sequence of the resulting genomic DNA, the transcriptional regulatory region of a cDNA disclosed in this invention can be predicted and obtained. The obtained transcriptional regulatory region is cloned so as to be localized upstream of a reporter gene to prepare a reporter construct. The resulting reporter construct is introduced into a cultured cell strain to prepare a transformant for screening. By contacting a candidate compound with this transformant to detect the expression of a reporter gene, it is possible to assess the effect of the candidate compound on the transcriptional regulatory region.

Based on the method of detecting the effect on the expression level of the polynucleotides of this invention, it is possible to carry out screening for a compound that alters the expression level of one or more polynucleotides. This invention relates to a method of screening for a compound that alters the expression level of a polynucleotide according to above-described (a) or (b), comprising following steps.

That is, the present invention relates to a method of screening for a compound that decreases the expression level of a polynucleotide of above-described (a) or (b), the method comprising the steps of detecting the effect of a candidate compound on the expression level of the polynucleotide in vivo and/or in vitro, and selecting a compound that raises the expression level as compared to a control.

Alternatively, this invention relates to a method of screening for a compound that acts on the transcriptional regulatory region by the reporter assay utilizing the transcriptional regulatory region of the gene comprising the nucleotide sequence of any one of SEQ ID NOs: 1 to 17. Based on the results of reporter assay according to this invention, by selecting a compound that decreases the expression level of the reporter gene as compared to a control, it is possible to obtain a compound that suppresses the expression of the gene comprising the nucleotide sequence of any one of SEQ ID NOs: 1 to 17.

The polynucleotide, antibody, cell line, or model animal, which are necessary for the various methods of screening of this invention, can be combined in advance to produce a kit. More specifically, such a kit may comprise, for example, a cell that expresses the indicator gene, and a reagent for measuring the expression level of the gene. As a reagent for measuring the expression level of the indicator gene, for example, an oligonucleotide that has at least 15 nucleotides complementary to the polynucleotide comprising the nucleotide sequence of at least one indicator gene or to the complementary strand thereof may be used. Alternatively, an antibody that recognizes a peptide comprising amino acid sequence of at least one indicator protein may be used as a reagent. In these kits may be packaged a substrate compound used for the detection of the indicator, medium and a vessel for cell culturing, positive and negative standard samples, and furthermore, a manual describing how to use the kit. A kit of this invention, for detecting the effect of a candidate compound on the expression level of the genes of this invention, can be used for screening for a compound that modifies the expression level of the genes of this invention.

Test candidate compounds used in these methods include, in addition to compound preparations synthesized by known chemical methods, steroid derivatives and compound preparations synthesized by combinatorial chemistry, and mixtures of multiple compounds such as extracts from animal or plant tissues, or microbial cultures and their purified preparations.

Compounds selected by the screening method of this invention are useful as therapeutic agents for an allergic disease. The expression levels of genes of this invention are increased in eosinophils of patients with early stage allergic diseases. Accordingly, a compound capable of increasing the expressions of the genes is expected to suppress symptoms of atopic dermatitis. A therapeutic agent for one or more allergic diseases of the present invention can be formulated by including a compound selected by the screening methods as the effective ingredient, and mixing with a physiologically acceptable carrier, excipient, diluent, and such. To ameliorate allergic symptoms, the therapeutic agent for allergic diseases of this invention can be administered orally or parenterally.

Oral drugs can take any dosage form including granules, powder, tablets, capsules, solution, emulsion, suspension, and so on. Injections include subcutaneous injection, intramuscular injection, and intraperitoneal injection.

Furthermore, for administering a compound that is composed of protein, a therapeutic effect can be achieved by introducing a gene encoding the protein into the living body using gene therapeutic techniques. The techniques for treating disease by introducing a gene encoding a therapeutically effective protein into the living body and expressing it therein are well known in the art.

Alternatively, an antisense DNA can be incorporated downstream of an appropriate promoter sequence to be administered as an antisense RNA expression vector. When this expression vector is introduced into eosinophils of an allergic disease patient, a therapeutic effect on allergic disease is achieved by the reduction of the expression level of the gene through the expression of the corresponding antisense gene. For introducing the expression vector into eosinophil cells, methods performed either in vivo or ex vivo are known.

Furthermore, compounds that inhibit the activity of proteins (i.e. indicator proteins) that are expression products of the indicator genes of this invention, are also expected to show therapeutic effects on allergic diseases. For example, antibodies that recognize the indicator proteins of this invention and suppress their activity are useful as pharmaceutical agents for treatment of allergic diseases. Methods for preparing antibodies that suppress protein activity are well known. For administration to humans, antibodies may be prepared as chimeric antibodies, humanized antibodies, or human-type antibodies to serve as highly safe pharmaceutical agents.

Although the dosage may vary depending on the age, sex, body weight, and symptoms of a patient; treatment effects; method for administration; treatment duration; type of active ingredient contained in the drug composition; and such, a range of 0.1 to 500 mg, preferably 0.5 to 20 mg per dose for an adult can be administered. However, the dose changes according to various conditions, and thus in some case a smaller amount than that mentioned above is sufficient whereas an amount above the above-mentioned range is required in other cases.

### BRIEF DESCRIPTION OF THE DRAWINGS

FIG. 1 is a graph showing the distribution of the numbers of peripheral blood eosinophils (cells/μL) in healthy subjects and in patients with various atopic dermatitis symptoms.

FIG. 2 is a graph showing the distribution of total IgE concentrations (UA/mL) in healthy subjects and in patients with various atopic dermatitis symptoms.

FIG. 3 is a graph showing the distribution of the 1858-05 gene expression levels (copy/ng RNA) in healthy subjects and in patients with various atopic dermatitis symptoms.

FIG. 4 is a graph showing the distribution of the 1901-21 gene expression levels (copy/ng RNA) in healthy subjects and in patients with various atopic dermatitis symptoms.

FIG. 5 is a graph showing the distribution of the 1913-17 gene expression levels (copy/ng RNA) in healthy subjects and in patients with various atopic dermatitis symptoms.

FIG. 6 is a graph showing the distribution of the 1852-09 gene expression levels (copy/ng RNA) in healthy subjects and in patients with various atopic dermatitis symptoms.

FIG. 7 is a graph showing the distribution of the 1945-03 gene expression levels (copy/ng RNA) in healthy subjects and in patients with various atopic dermatitis symptoms.

FIG. 8 is a graph showing the distribution of the 1948-16 gene expression levels (copy/ng RNA) in healthy subjects and in patients with various atopic dermatitis symptoms.

FIG. 9 is a graph showing the distribution of the 1833-02 gene expression levels (copy/ng RNA) in healthy subjects and in patients with various atopic dermatitis symptoms.

FIG. 10 is a graph showing the distribution of the 1873-30 gene expression levels (copy/ng RNA) in healthy subjects and in patients with various atopic dermatitis symptoms.

FIG. 11 is a graph showing the distribution of the 1937-03 gene expression levels (copy/ng RNA) in healthy subjects and in patients with various atopic dermatitis symptoms.

FIG. 12 is a graph showing the distribution of the 1949-02 gene expression levels (copy/ng RNA) in healthy subjects and in patients with various atopic dermatitis symptoms.

FIG. 13 is a graph showing the distribution of the 1956-04 gene expression levels (copy/ng RNA) in healthy subjects and in patients with various atopic dermatitis symptoms.

FIG. 14 is a graph showing the distribution of the 1919-13 gene expression levels (copy/ng RNA) in healthy subjects and in patients with various atopic dermatitis symptoms.

FIG. 15 is a graph showing the distribution of the 1917-03 gene expression levels (copy/ng RNA) in healthy subjects and in patients with various atopic dermatitis symptoms.

FIG. 16 is a graph showing the distribution of the 1941-20 gene expression levels (copy/ng RNA) in healthy subjects and in patients with various atopic dermatitis symptoms.

FIG. 17 is a graph showing the distribution of the 1930-03 gene expression levels (copy/ng RNA) in healthy subjects and in patients with various atopic dermatitis symptoms.

FIG. 18 is a graph showing the distribution of the 1921-05 gene expression levels (copy/ng RNA) in healthy subjects and in patients with various atopic dermatitis symptoms.

FIG. 19 is a graph showing the distribution of the 1925-08 gene expression levels (copy/ng RNA) in healthy subjects and in patients with various atopic dermatitis symptoms.

FIG. 20 is a graph showing the expression levels of the 1858-05 gene (copy/ng RNA, GAPDH corrected value) in the peripheral blood eosinophils of a healthy subject when the cells were cultured in the presence of various cytokines indicated along the horizontal axis.

FIG. 21 is a graph showing the expression level of the 1901-21 gene under the same conditions as in FIG. 20.

FIG. 22 is a graph showing the expression level of the 1913-17 gene under the same conditions as in FIG. 20.

FIG. 23 is a graph showing the expression level of the 1852-09 gene under the same conditions as in FIG. 20.

FIG. 24 is a graph showing the expression level of the 1945-03 gene under the same conditions as in FIG. 20.

FIG. 25 is a graph showing the expression level of the 1948-16 gene under the same conditions as in FIG. 20.

FIG. 26 is a graph showing the expression level of the 1833-02 gene under the same conditions as in FIG. 20.

FIG. 27 is a graph showing the expression level of the 1873-30 gene under the same conditions as in FIG. 20.

FIG. 28 is a graph showing the expression level of the 1949-02 gene under the same conditions as in FIG. 20.

FIG. 29 is a graph showing the expression level of the 1956-04 gene under the same conditions as in FIG. 20.

FIG. 30 is a graph showing the expression level of the 1919-13 gene under the same conditions as in FIG. 20.

FIG. 31 is a graph showing the expression level of the 1917-13 gene under the same conditions as in FIG. 20.

FIG. 32 is a graph showing the expression level of the 1941-20 gene under the same conditions as in FIG. 20.

FIG. 33 is a graph showing the expression level of the 1930-03 gene under the same conditions as in FIG. 20.

FIG. 34 is a graph showing the expression level of the 1921-05 gene under the same conditions as in FIG. 20.

FIG. 35 is a graph showing the expression level of the 1925-08 gene under the same conditions as in FIG. 20.

### BEST MODE FOR CARRYING OUT THE INVENTION

The present invention is explained in detail below with reference to examples, but should not to be construed as being limited thereto.

### EXAMPLE 1

### Differential Display Analysis

Screening was performed to discover novel genes relating to therapy or useful for diagnosis, which show varying expression when comparing hemocytes isolated from the peripheral blood of a healthy patient to those of an atopic dermatitis patient.

### (1) Subjects

Symptoms, pathology, presence of asthma, mite-specific IgE values, numbers of eosinophils, and total IgE values of healthy subjects (lanes 1 to 6) and those with atopic dermatitis (lanes 8 to 29) are shown in Table 1. Allergen non-specific (Total IgE) mite-specific, and cedar-specific IgEs were measured by the EIA method. More specifically, the test sera were allowed to react to an anti-human IgE antibody-bound cap to bind thereto allergen non-specific IgE antibody or mite- or cedar-specific IgE antibodies in the sera. Next, β-D-galactosidase-labeled anti-human IgE antibody and a substrate solution (4-methylumbelliferyl-β-D-galactopyranoside) were added and allowed to react to produce a fluorescent substance. The reaction was quenched by adding a quenching solution, and the antibody concentration was determined from the fluorescence intensity of a simultaneously measured standard IgE. LDH was measured by the UV method (Wroblewski-La Due method) and the rate of decrease of NADH caused by the reaction of pyruvic acid with NADH is calculated from decrease in absorbance. L-type Wako LDH (Wako Pure Chemicals) and 7170-type automatic analyzer (Hitachi) were used for measuring the LDH values. The number of eosinophils was measured by microscopic examination and automatic hemocyte analyzer SE-9000 (RF/DC impedance system, Sysmex) using 2 ml of EDTA-added blood as the sample.

TABLE 1

| | Lane | | | | | | | | | | | | | | | | | | |
|---|---|---|---|---|---|---|---|---|---|---|---|---|---|---|---|---|---|---|---|
| | 1 | 2 | 3 | 4 | 5 | 6 | 8 | 9 | 10 | 11 | 12 | 14 | 18 | 19 | 24 | 26 | 27 | 28 | 29 |
| Blood | 120 | 140 | 19 | 20 | 24 | 25 | 36 | 43 | 69 | 90 | 73 | 92 | 56 | 59 | 30 | 46 | 48 | 51 | 60 |
| Symptom | Healthy subject, | | | | | | Light | | | | Moderate | | | | Severe | | | | |
| | very light | | | | | | | | | | | | | | | | | | |
| Pathology | | | | | | | ∘ | ∘ | ∘ | ∘ | ∘ | ∘ | • | • | • | • | • | • | • |
| Asthma | | | | | | | Light | Light | None | Light | None | None | None | Light | None | None | None | Light | None |
| S IgE | − | − | − | − | − | − | + | + | + | + | + | + | + | + | + | + | + | + | + |
| Eosinophil | B | B | B | B | B | A | B | C | C | C | C | C | B | C | C | C | C | B | C |
| T IgE | L | L | L | L | L | L | L | L | L | H | H | L | H | H | L | L | H | H | H |

In the above table, “o” and “ ” for pathology refer to the remission stage and increment stage, respectively. For specific IgE (S IgE), Classes 0 to 2 and Classes 3 to 6 of anti-mite IgEs were denoted as “−” and “+”, respectively. For total IgE (T IgE), 1000 IU/mL or less was referred to as “Low (L)”, and greater than 1000 IU/ml as “High (H) ”. For eosinophils, less than 3% was denoted as “A”, 3% to 7% as “B”, and greater than 7% as “C”.

### (2) Differential Display Analysis

A 3% dextran solution was added to whole blood drawn from a healthy subject and a patient, and this was left to stand at room temperature for 30 minutes to precipitate erythrocytes. The upper layer leukocyte fraction was collected, layered on top of Ficoll solution (Ficoll-Paque PLUS; Amersham Pharmacia Biotech), and centrifuged at 1500 rpm for 30 minutes at room temperature. The granulocyte fraction that collected in the lower layer was reacted with CD16 antibody magnetic beads at 4° C. for 30 minutes, and cells that had eluted without being trapped in the separation using MACS were used in the experiment as eosinophils.

Eosinophils prepared as described above were dissolved in Isogen (Nippon Gene; Wako Pure Chemicals), and from this solution, RNA was separated according to the protocol attached to Isogen. Chloroform was added, the mixture was stirred and centrifuged, and the aqueous layer was collected. Next, isopropanol was added, the mixture was stirred and centrifuged, and the precipitated total RNA was collected. DNase (Nippon Gene; Wako Pure Chemicals) was added to the collected total RNA, the mixture was reacted at 37° C. for 15 minutes, and RNA was collected by phenol-chloroform extraction followed by ethanol precipitation.

Fluorescent Differential Display (abbreviated to DD) analysis using total RNA thus prepared was carried out according to the literature (T. Ito et al., 1994, FEBS Lett. 351: 231-236). The total RNA was reverse transcribed to obtain cDNA. In the first DD-PCR, 0.2 μg each of total RNA was used for three types of anchor primers to synthesize cDNAs. In the second DD-PCR, 0.4 μg each of total RNA was used for the synthesis of cDNAs using three types of anchor primers. In both cases, the cDNAs were diluted to a final concentration equivalent to 0.4 ng/μl RNA and used for further experiments. The DD-PCR was carried out using an amount of CDNA equivalent to 1 ng RNA per reaction. The reaction mixture composition is shown in Table 2.

TABLE 2

| | |
|---|---|
| cDNA (equivalent to 0.4 ng/μl RNA) | 2.5 μl |
| Arbitrary primer (2 μM) | 2.5 μl |
| 10x AmpliTaq PCR buffer | 1.0 μl |
| 2.5 mM dNTP | 0.8 μl |
| 50 μM anchor primer | 0.1 μl |
| (GT15A, GT15C, or GT15G) | |
| Gene Taq (5 U/μl) | 0.05 μl |
| AmpliTaq (5 U/μl) | 0.05 μl |
| dH₂O | 3.0 μl |
| Total volume | 10.0 μl |

The PCR was carried out at following condition: 1 cycle of “95° C. for 3 min, 40° C. for 5 min, and 72° C. for 5 min”; subsequently 30 cycles of “94° C. for 15 sec, 40° C. for 2 min, and 72° C. for 1 min”; after these cycles, 72° C. for 5 min; and then continuously 4° C.

Reactions were conducted using 287 primer pairs: i.e., anchor primers GT15A (SEQ ID NO: 18), GT15C (SEQ ID NO: 19), and GT15G (SEQ ID NO: 20) were used in combination with arbitrary primers AG 1 to AG 110, AG 111 to AG 199, and AG 200 to AG 287, respectively. As for the arbitrary primers, oligomers having l0 nucleotides with a GC content of 50% were designed and synthesized.

For gel electrophoresis, a 6% denaturing polyacrylamide gel was prepared, and 2.5 μl sample from the PCR was applied and run under 40 W for 210 min. After electrophoresis, the gel was scanned by Hitachi fluorescence imaging analyzer FMBIO II, and the gel image was obtained by detecting fluorescence.

Samples from both healthy subjects and patients were electrophoresed side-by-side, and the bands that showed variation in expression between each of the samples were isolated. Sequences were determined for the bands that were selected by visual judgment and indicated values of 0.1 or less in significance tests. Furthermore, sequences were determined for bands selected using an image analysis software, Bio-Image. Identical sequence clones in each of the bands were grouped, and were designated as consensus sequences. As a result, among the sequence determined bands, a band that can be uniquely defined as the “dominant sequence” was selected.

The selected consensus sequence was used as the query to perform a homology search through genembl and dbEST using BLAST in GCG. Herein, a sequence with 95% or more identity was determined as the sequence “with significant homology”.

As a result of such analysis, bands that showed increased expression specifically in patients were identified. The primer sets used to amplify each of the identified bands are shown in Table 3. The number in parenthesis after the sequence of the arbitrary primer is the SEQ ID NO. Furthermore, the nucleotide sequences of each of the bands are as shown in the following SEQ ID NOS.

“1858-05”/SEQ ID NO: 1

“1901-21”/SEQ ID NO: 101

“1913-17”/SEQ ID NO: 102

“1852-09”/SEQ ID NO: 103

“1945-03”/SEQ ID NO: 104

“1948-16”/SEQ ID NO: 6

“1833-02”/SEQ ID NO: 7

“1873-30”/SEQ ID NO: 8

“1937-03”/SEQ ID NO: 9

“1949-02”/SEQ ID NO: 10

“1956-04”/SEQ ID NO: 105

“1919-13”/SEQ ID NO: 12

“1917-03”/SEQ ID NO: 13

“1941-20”/SEQ ID NO: 14

“1930-03”/SEQ ID NO: 15

“1921-05”/SEQ ID NO: 16

“1925-08”/SEQ ID NO: 17

TABLE 3

| | | | Name of | Sequence of |
|---|---|---|---|---|
| | | Anchor | arbitrary | arbitrary primer |
| Band ID | bp | primer | primer | (SEQ ID NO) |
| 1858-05 | 231 | GT15C | AG00140 | TTGATGGACC (21) |
| 1901-21 | 189 | GT15G | AG00262 | TATTGCCGTG (22) |
| 1913-17 | 197 | GT15G | AG00278 | GACGGTTAGT (23) |
| 1852-09 | 185 | GT15A | AG00029 | GGACTTCGTA (24) |
| 1945-03 | 283 | GT15G | AG00264 | GGAAGAGTTG (25) |
| 1948-16 | 210 | GT15G | AG00260 | ATCCGTACTG (26) |
| 1833-02 | 293 | GT15A | AG00016 | TCCCTACAGA (27) |
| 1873-30 | 574 | GT15C | AG00161 | TCATAGTCCG (28) |
| 1937-03 | 527 | GT15G | AG00251 | AAACCCATCG (29) |
| 1949-02 | 437 | GT15G | AG00262 | TATTGCCGTG (30) |
| 1956-04 | 252 | GT15G | AG00275 | CAGAACTGCT (31) |
| 1919-13 | 321 | GT15G | AG00213 | AAGGAACGGA (32) |
| 1917-03 | 328 | GT15G | AG00202 | ATGGGAGGAA (33) |
| 1941-20 | 274 | GT15G | AG00265 | GCTGGTTTTG (34) |
| 1930-03 | 347 | GT15G | AG00235 | GTTTGCTTGC (35) |
| 1921-05 | 784 | GT15G | AG00219 | CACGAGTCTA (36) |
| 1925-08 | 224 | GT15G | AG00220 | GAGCAAGGTA (37) |

### EXAMPLE 2

### Quantification of Expression Level by ABI 7700

The expression of genes identified in Example 1 was analyzed by TaqMan method using ABI 7700. RNAs were prepared in the same manner as in Example 1 from 10 samples each of freshly collected eosinophils from healthy subjects and patients with light, moderate, and severe atopic dermatitis. The examination value profiles of healthy subjects and patients are shown in Table 4. Expression levels were quantified for the gene in the band identified in Example 1, and for β-actin gene, which is known to be an internal standard for correction.

TABLE 4

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
|---|---|---|---|---|---|---|---|---|---|---|---|---|---|---|---|---|---|---|---|---|---|---|---|
| Blood | | | | | | | | | | | 80 | 109 | 125 | 130 | 131 | 164 | 170 | 197 | 205 | 215 | 101 | 147 | 162 |
| Symp- | None | | | | | | | | | | Light | | | | | | | | | | Moderate | | |
| tom | | | | | | | | | | | | | | | | | | | | | | | |
| Pathol- | None | | | | | | | | | | ∘ | ∘ | ∘ | ∘ | ∘ | ∘ | ∘ | ∘ | ∘ | ∘ | ∘ | ∘ | ∘ |
| ogy | | | | | | | | | | | | | | | | | | | | | | | |
| Asthma | None | | | | | | | | | | Light | None | None | Light | Light | Light | None | Light | None | Light | None | None | None |
| Mite | − | | | | | | | | | | + | + | + | + | + | + | + | + | + | + | + | + | + |
| IgE | | | | | | | | | | | | | | | | | | | | | | | |
| Eosin- | B | B | B | A | B | B | B | A | A | B | C | B | B | C | C | C | C | C | C | C | B | C | C |
| ophil | | | | | | | | | | | | | | | | | | | | | | | |

| | | | | | | | | | | | | | | |
|---|---|---|---|---|---|---|---|---|---|---|---|---|---|---|
| Total | L | L | L | H | H | H | L | L | L | L | L | H | L | H |
| IgE | | | | | | | | | | | | | | |

| | 24 | 25 | 26 | 27 | 28 | 29 | 30 | 31 | 32 | 33 | 34 | 35 | 36 | 37 | 38 | 39 | 40 |
|---|---|---|---|---|---|---|---|---|---|---|---|---|---|---|---|---|---|
| Blood | 179 | 196 | 210 | 218 | 219 | 226 | 232 | 96 | 135 | 146 | 167 | 184 | 194 | 211 | 225 | 227 | 238 |
| Symp- | Moderate | | | | | | | Severe | | | | | | | | | |
| tom | | | | | | | | | | | | | | | | | |
| Pathol- | • | • | • | • | ∘ | ∘ | ∘ | • | • | • | • | • | • | • | • | • | • |
| ogy | | | | | | | | | | | | | | | | | |
| Asthma | Light | None | Light | None | None | None | None | None | None | None | Light | None | None | Light | None | None | None |
| Mite | + | + | + | + | + | + | − | + | + | + | + | + | + | + | + | + | + |
| IgE | | | | | | | | | | | | | | | | | |
| Eosin- | B | A | C | C | B | C | A | B | C | B | B | B | C | C | C | C | C |
| ophil | | | | | | | | | | | | | | | | | |
| Total | H | L | H | H | L | L | L | L | H | H | L | L | H | H | H | H | H |
| IgE | | | | | | | | | | | | | | | | | |

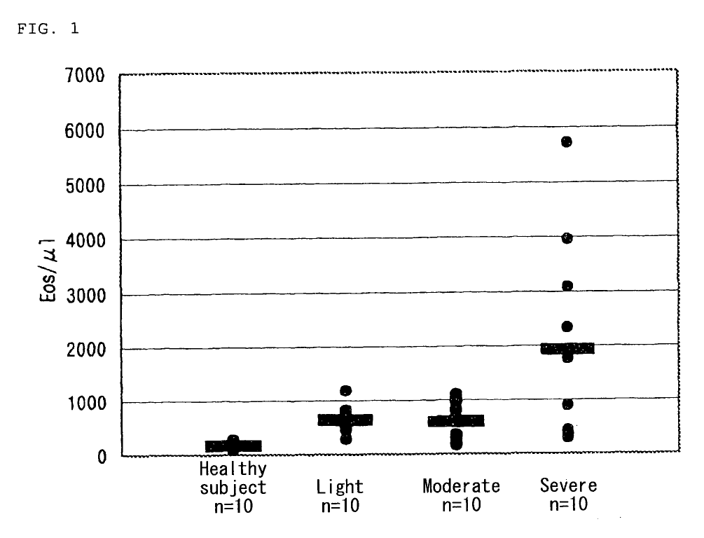

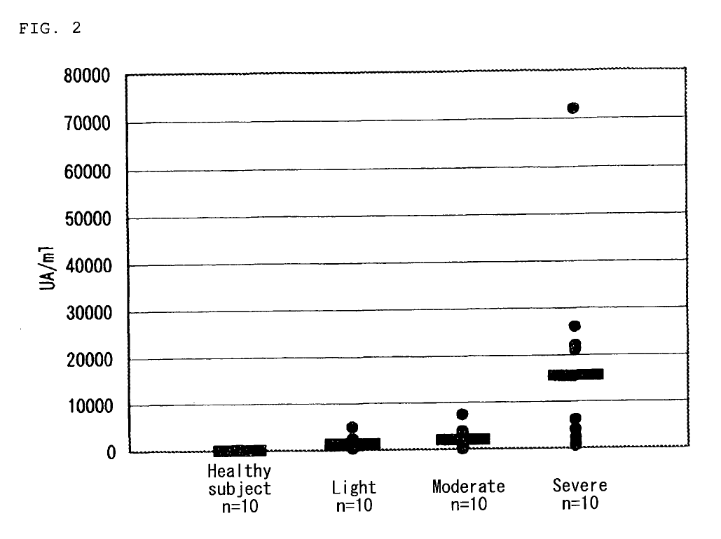

FIG. 1 (number of eosinophils) and FIG. 2 (total IgE) show plotted examination values of each group based on the examination value profiles of 10 samples each of healthy subjects and patients with light, moderate, and severe atopic dermatitis. The number of eosinophils in atopic dermatitis patients is evaluated as B to C, but when the actual measured values are compared, the measured values This shows that the number of eosinophils is difficult to utilize as an indicator for diagnosis of light or moderate atopic dermatitis.

A similar trend can be observed for the measured values of total IgE (FIG. 2). Specifically, marked increase in total IgE value is observed in severe patients, and a large difference compared to values in healthy subjects is not observed in light to moderate patients. Therefore, this shows that total IgE value is also difficult to use as an indicator for a light allergic disease.

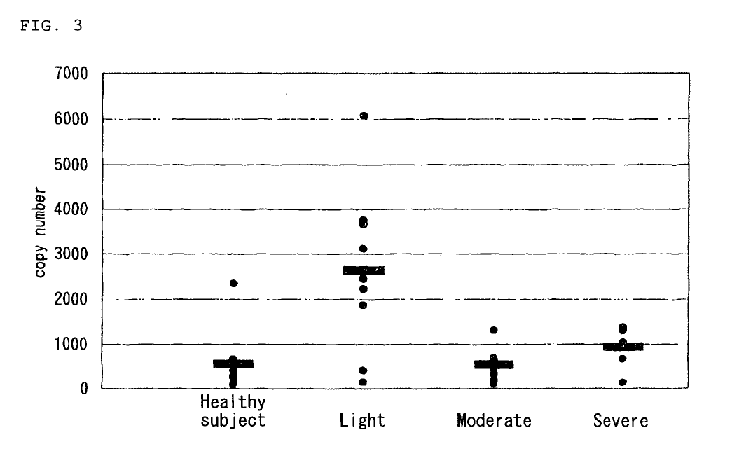

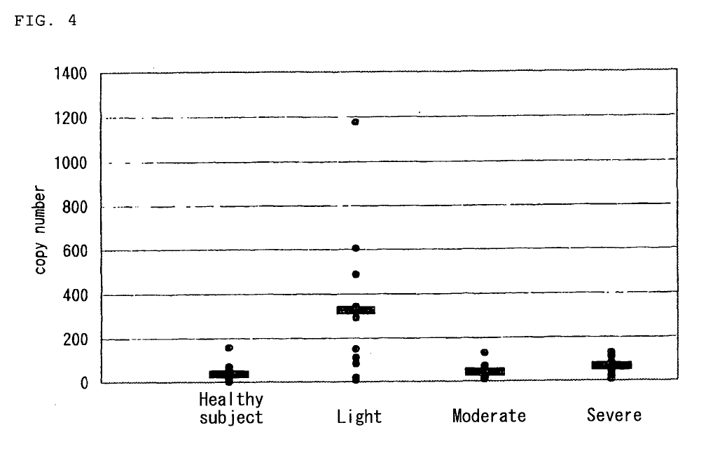

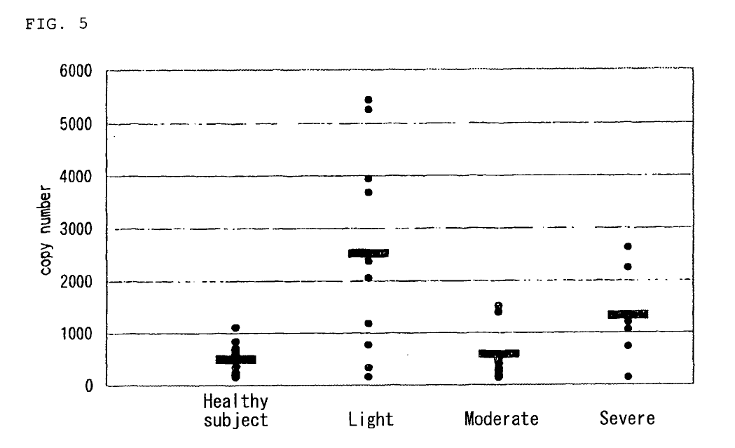

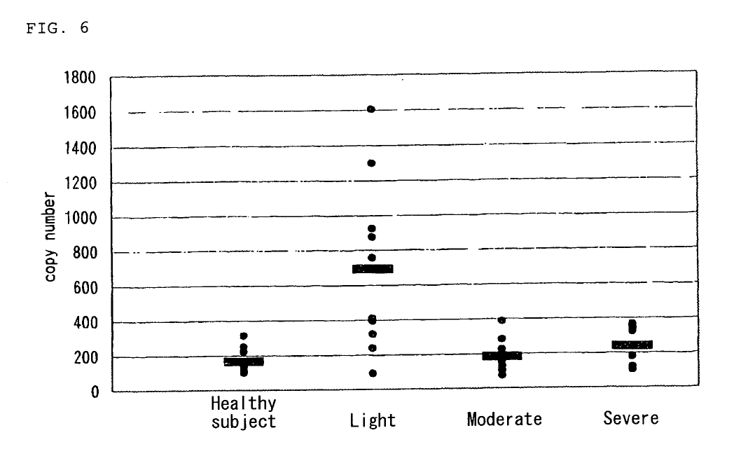

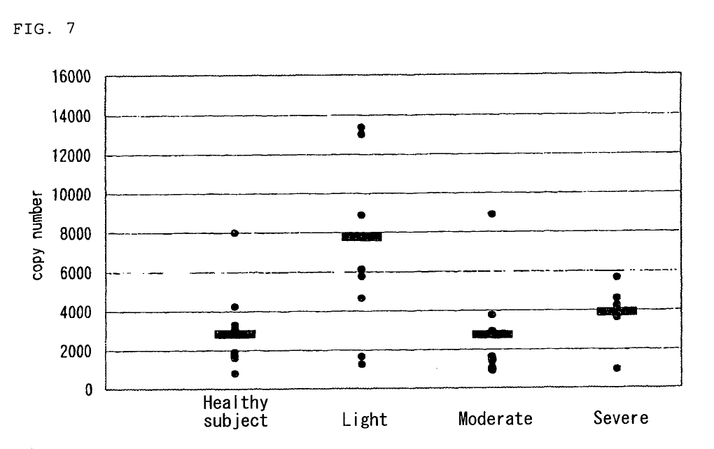

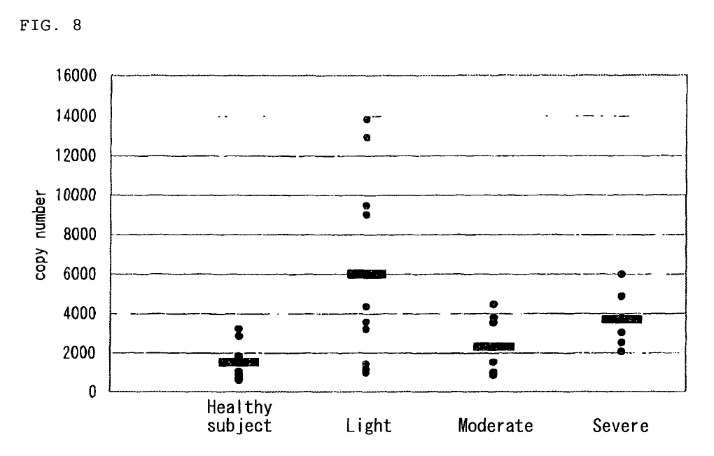

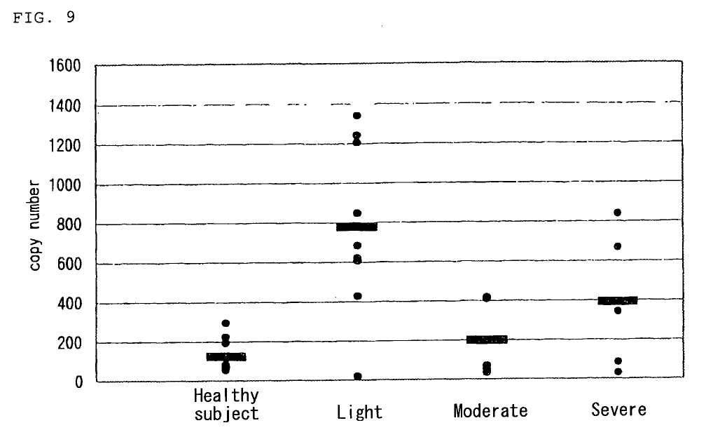

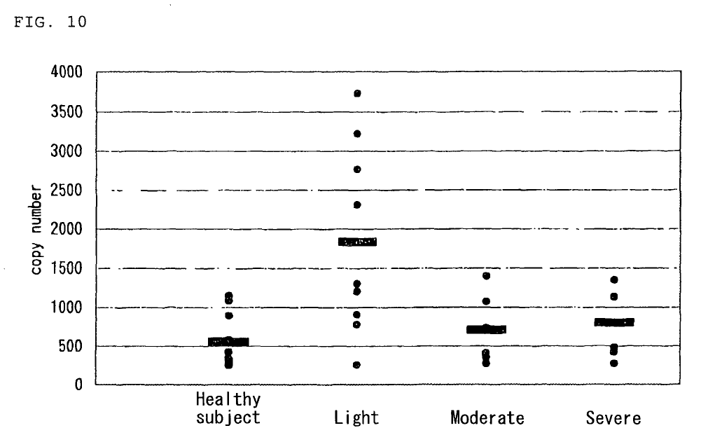

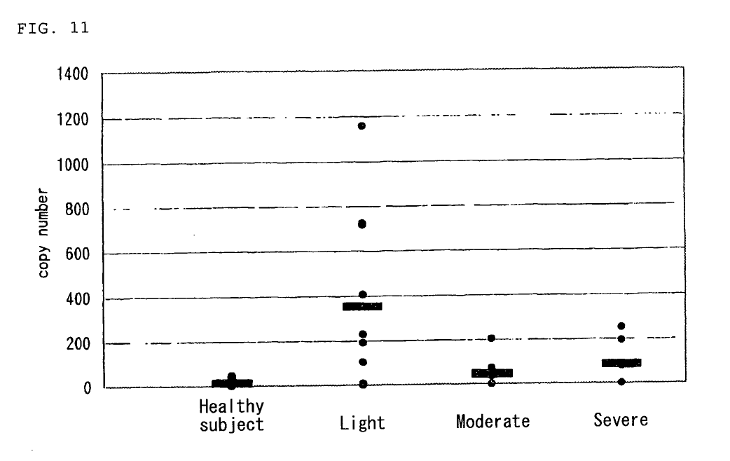

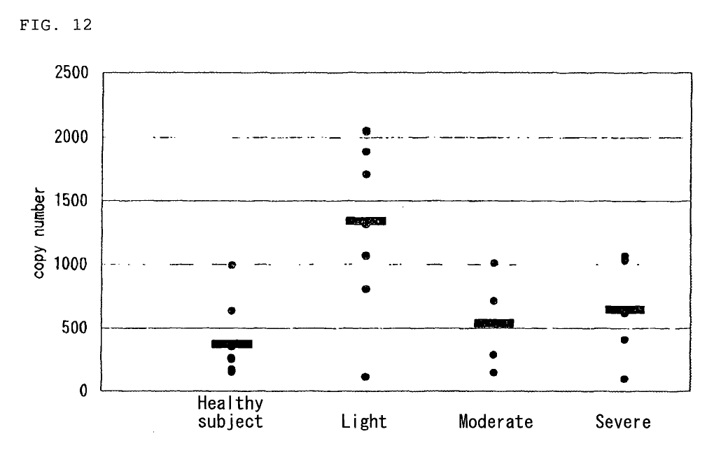

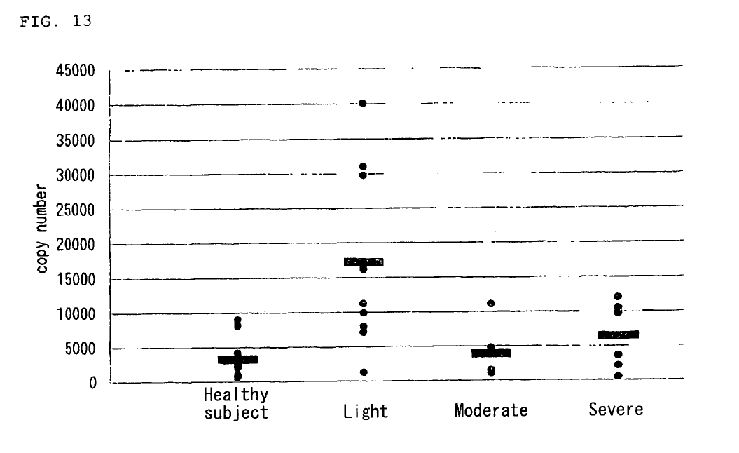

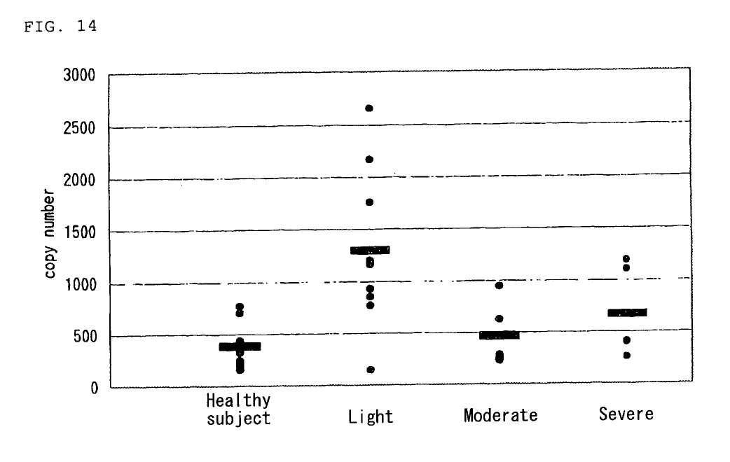

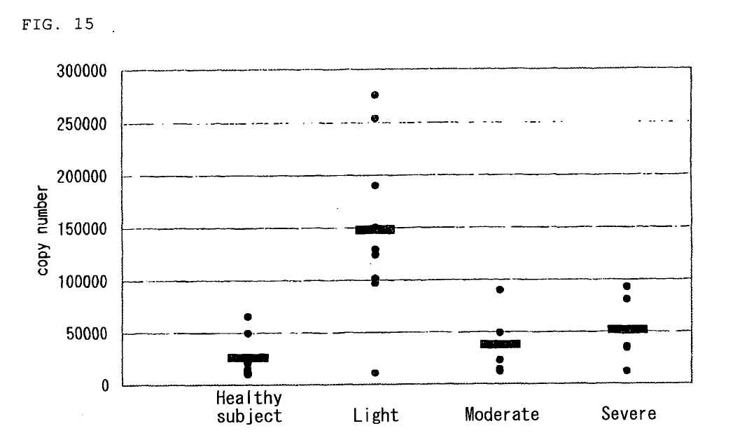

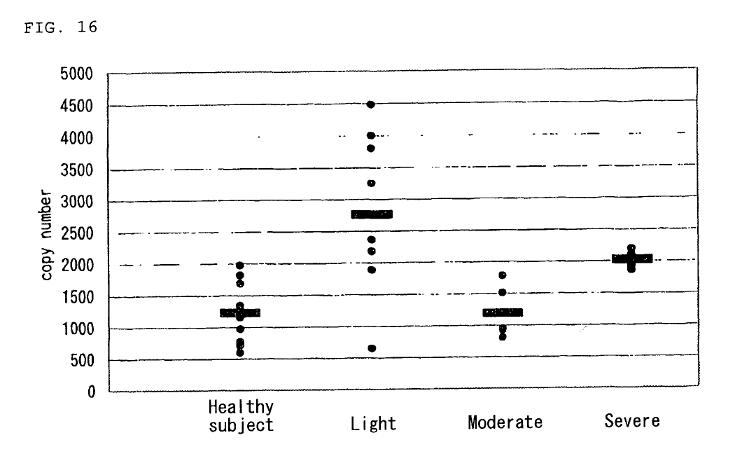

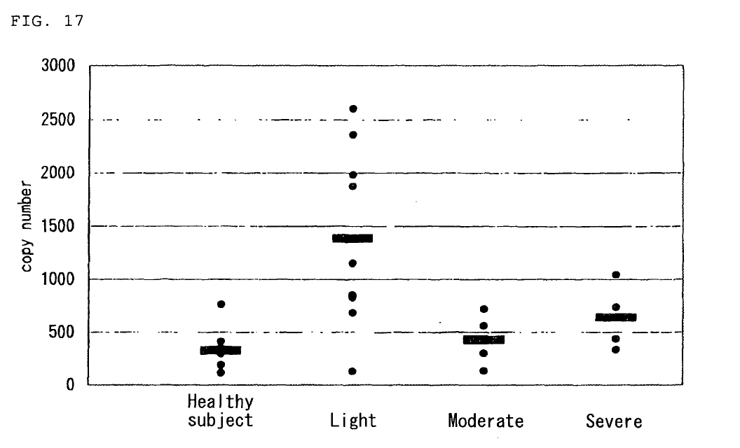

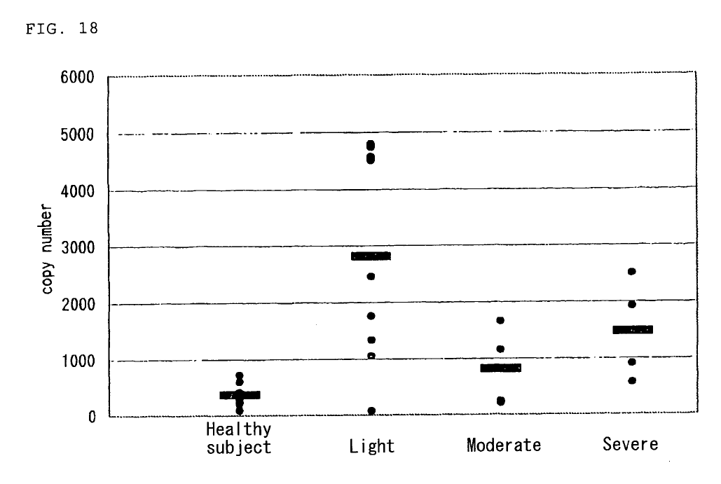

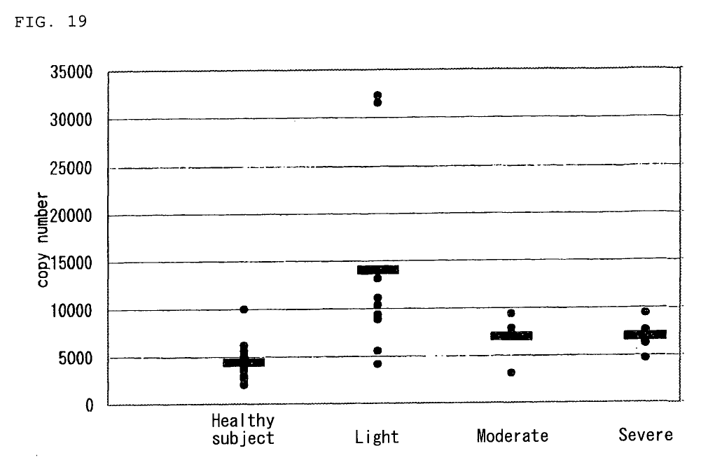

The primers and TaqMan probes used for measurements by ABI 7700 were designed by Primer Express (PE Biosystems) based on the sequence information for each gene. TaqMan probes are labeled on the 5′-end with FAM (6-carboxy-fluorescein), and on the 3 ′-end with TAMRA (6-carboxy-N,N,N′,N′-tetramethylrhodamine). The nucleotide sequences of the primers and the TaqMan probes used for the experiment are as shown in the respective SEQ ID NOs of Table 5. Primers and probes used for measuring β-actin were those included in TaqMan β-actin Control Reagents (PE Biosystems). The results of measurement are shown in FIG. 3 to FIG. 19. Furthermore, average expression levels are summarized in Table 6.

TABLE 5

| ID | Forward | Reverse | Probe |
|---|---|---|---|
| 1858-05 | 38 | 39 | 40 |
| 1901-21 | 41 | 42 | 43 |
| 1913-17 | 44 | 45 | 46 |
| 1852-09 | 47 | 48 | 49 |
| 1945-03 | 50 | 51 | 52 |
| 1948-16 | 53 | 54 | 55 |
| 1833-02 | 56 | 57 | 58 |
| 1873-30 | 59 | 60 | 61 |
| 1937-03 | 62 | 63 | 64 |
| 1949-02 | 65 | 66 | 67 |
| 1956-04 | 68 | 69 | 70 |
| 1919-13 | 71 | 72 | 73 |
| 1917-03 | 74 | 75 | 76 |
| 1941-20 | 77 | 78 | 79 |
| 1930-03 | 80 | 81 | 82 |
| 1921-05 | 83 | 84 | 85 |
| 1925-08 | 86 | 87 | 88 |
| β-actin | 89 | 90 | 91 |

TABLE 6
Expression level of genes in clinical samples
(AVERAGE: copy/ng (corrected value))

| | Healthy | | | |
|---|---|---|---|---|
| Band ID | people | Light | Moderate | Severe |
| 1858-05 | 561 | 2638 | 537 | 928 |
| 1901-21 | 36 | 326 | 41 | 65 |
| 1913-17 | 496 | 2515 | 585 | 1326 |
| 1852-09 | 165 | 691 | 182 | 238 |
| 1945-03 | 2820 | 7764 | 2747 | 3884 |
| 1948-16 | 1517 | 5993 | 2295 | 3692 |
| 1833-02 | 123 | 775 | 197 | 392 |
| 1873-30 | 553 | 1828 | 701 | 797 |
| 1937-03 | 14 | 354 | 45 | 87 |
| 1949-02 | 372 | 1340 | 538 | 645 |
| 1956-04 | 3228 | 17124 | 3862 | 6372 |
| 1919-13 | 386 | 1293 | 466 | 671 |
| 1917-03 | 26027 | 147863 | 37786 | 51244 |
| 1941-20 | 1222 | 2754 | 1191 | 2016 |
| 1930-03 | 329 | 1383 | 427 | 637 |
| 1921-05 | 365 | 2812 | 816 | 1474 |
| 1925-08 | 4385 | 14041 | 6961 | 6962 |

Using the above-mentioned data, parametric multiple comparison test and non-parametric multiple comparison test were carried out. Statistical analysis was carried out using SAS Pre-clinical Package of The SAS SYSTEM, Version 4.0 (SAS Institute Inc.). The results are shown in Table 7.

As apparent from Table 7, expression of each of the genes identified in the present invention was significantly increased due to atopic dermatitis (light). Therefore, this fact gives support to the diagnostic value of measuring the expression of these genes for atopic dermatitis.

TABLE 7

| | | | | | Nonparametric multiple | | | |
|---|---|---|---|---|---|---|---|---|
| Band | Parametric multiple comparison | | | | comparison | | | |
| ID | Dunnet P value | | Tukey P value | | Dunnet P value | | Tukey P value | |
| 1858-05 | Light > | 0.0003 | Light > | 0.0005 | Light > | 0.0052 | Light > | 0.0097 |
| | Normal | | Normal | | Normal | | Normal | |
| | | | Light > | 0.001 | | | Light > | 0.0449 |
| | | | Moderate | | | | Moderate | |
| | | | Light > | 0.0163 | | | | |
| | | | Severe | | | | | |
| 1901-21 | Light > | 0.009 | Light > | 0.016 | Light > | 0.0191 | Light > | 0.0343 |
| | Normal | | Normal | | Normal | | Normal | |
| | | | Light > | 0.0261 | | | | |
| | | | Moderate | | | | | |
| 1913-17 | Light > | 0.0019 | Light > | 0.0035 | Light > | 0.0212 | Light > | 0.0378 |
| | Normal | | Normal | | Normal | | Normal | |
| | | | Light > | 0.0092 | | | | |
| | | | Moderate | | | | | |
| 1852-09 | Light > | 0.0007 | Light > | 0.0012 | Light > | 0.0075 | Light > | 0.0139 |
| | Normal | | Normal | | Normal | | Normal | |
| | | | Light > | 0.0033 | | | Light > | 0.0243 |
| | | | Moderate | | | | Moderate | |
| | | | Light > | 0.0196 | | | | |
| | | | Severe | | | | | |
| 1945-03 | Light > | 0.0051 | Light > | 0.0092 | Light > | 0.0396 | Light > | 0.0208 |
| | Normal | | Normal | | Normal | | Moderate | |
| | | | Light > | 0.0134 | | | | |
| | | | Moderate | | | | | |
| 1948-16 | Light > | 0.0049 | Light > | 0.0089 | Light > | 0.0174 | | |
| | Normal | | Normal | | Normal | | | |
| | | | Light > | 0.0342 | Severe > | 0.0516 | | |
| | | | Moderate | | Normal | | | |
| 1833-02 | Light > | 0.0004 | Light > | 0.0007 | Light > | 0.01 | Light > | 0.0182 |
| | Normal | | Normal | | Normal | | Normal | |
| | | | Light > | 0.0112 | | | | |
| | | | Moderate | | | | | |
| 1873-30 | Light > | 0.0026 | Light > | 0.0047 | Light > | 0.0174 | Light > | 0.031 |
| | Normal | | Normal | | Normal | | Normal | |
| | | | Light > | 0.0368 | | | | |
| | | | Moderate | | | | | |
| 1937-03 | Light > | 0.0064 | Light > | 0.0115 | Light > | 0.0198 | Light > | 0.0354 |
| | Normal | | Normal | | Normal | | Normal | |
| | | | Light > | 0.0458 | | | | |
| | | | Moderate | | | | | |
| 1949-02 | Light > | 0.0007 | Light > | 0.0013 | Light > | 0.0065 | Light > | 0.0119 |
| | Normal | | Normal | | Normal | | Normal | |
| | | | Light > | 0.0443 | | | | |
| | | | Moderate | | | | | |
| 1956-04 | Light > | 0.0025 | Light > | 0.0046 | Light > | 0.0071 | Light > | 0.013 |
| | Normal | | Normal | | Normal | | Normal | |
| | | | Light > | 0.0314 | | | | |
| | | | Moderate | | | | | |
| 1919-13 | Light > | 0.0018 | Light > | 0.0032 | Light > | 0.0099 | Light > | 0.0179 |
| | Normal | | Normal | | Normal | | Normal | |
| | | | Light > | 0.0328 | | | | |
| | | | Moderate | | | | | |
| 1917-03 | Light > | 0.0001 | Light > | 0.0003 | Light > | 0.0029 | Light > | 0.0054 |
| | Normal | | Normal | | Normal | | Normal | |
| | | | Light > | 0.0049 | | | | |
| | | | Moderate | | | | | |
| | | | Light > | 0.0146 | | | | |
| | | | Severe | | | | | |
| 1941-20 | Light > | 0.0011 | Light > | 0.0019 | Light > | 0.0039 | Light > | 0.0073 |
| | Normal | | Normal | | Normal | | Normal | |
| | | | Light > | 0.0078 | | | Light > | 0.0267 |
| | | | Moderate | | | | Moderate | |
| 1930-03 | Light > | 0.0024 | Light > | 0.0043 | Light > | 0.0066 | Light > | 0.0122 |
| | Normal | | Normal | | Normal | | Normal | |
| | | | Light > | 0.0436 | | | | |
| | | | Moderate | | | | | |
| 1921-05 | Light > | 0.0015 | Light > | 0.0028 | Light > | 0.007 | Light > | 0.0128 |
| | Normal | | Normal | | Normal | | Normal | |
| 1925-08 | Light > | 0.0069 | Light > | 0.0121 | Light > | 0.0017 | Light > | 0.0032 |
| | Normal | | Normal | | Normal | | Normal | |

### EXAMPLE 3

### Expression of Genes of This Invention in Various Blood Cells

Expression of each gene was examined in cells separated from peripheral blood collected from five normal healthy subjects. Separation of eosinophils (E) was carried out as described above. After the elution of eosinophils, neutrophils (N) were prepared by releasing the cells, which were trapped with CD16 antibody magnetic beads, from the magnetic field, eluting, and recovering. On the other hand, the monocyte fraction recovered in the middle layer by the Ficoll-centrifugation was separated into the fraction eluted from MACS CD3 antibody magnetic beads (mixture of M (monocyte) and B cell) and fraction trapped therein (T-cell fraction). Then, using MACS CD14 antibody magnetic beads, the eluted fraction was separated into the eluted fraction (B cell fraction) and trapped fraction (monocyte fraction), and those three fractions were referred to as the purified T cells, B cells, and monocytes.

Eosinophils were solubilized using Isogen, while neutrophils, T cells, B cells and monocytes were solubilized with RNeasy (Qiagen), and total RNA were extracted, treated with DNase (by the same methods as described above), and subjected to the gene expression analysis. Primers, probes, and others used were the same as above. Average expression levels (AVERAGE: copying (corrected value)) in these blood cells are shown in Table 8.

TABLE 8
Expression level of genes in various blood cells
(AVERAGE: copying (corrected value))

| Band ID | Eosinophil | Neutrophil | B cell | T cell | Monocyte |
|---|---|---|---|---|---|
| 1858-05 | 2018 | 571 | 66 | 42 | 18 |
| 1901-21 | 366 | 11 | 163 | 252 | 9 |
| 1913-17 | 2567 | 3954 | 323 | 548 | 259 |
| 1852-09 | 774 | 165 | 30 | 12 | 68 |
| 1945-03 | 4489 | 7576 | 274 | 347 | 307 |
| 1948-16 | 4308 | 14307 | 3053 | 2001 | 775 |
| 1833-02 | 346 | 54 | 18 | 49 | 9 |
| 1873-30 | 1447 | 5675 | 1190 | 1592 | 321 |
| 1937-03 | 346 | 3 | 5 | 5 | 5 |
| 1949-02 | 1160 | 114 | 691 | 817 | 253 |
| 1956-04 | 5457 | 2197 | 520 | 903 | 151 |
| 1919-13 | 1371 | 233 | 189 | 311 | 106 |
| 1917-03 | 72621 | 47903 | 11135 | 8707 | 10897 |
| 1941-20 | 3166 | 189 | 422 | 464 | 184 |
| 1930-03 | 682 | 155 | 66 | 106 | 28 |
| 1921-05 | 1757 | 1323 | 82 | 150 | 38 |
| 1925-08 | 12535 | 8302 | 883 | 473 | 1532 |

### EXAMPLE 4

### Elongation of the Genetic Nucleotide Sequence (1901-21)

Using Marathon cDNA Amplification Kit (CLONTECH), PCR was carried out with Human Leukocyte Marathon-Ready cDNA (CLONTECH) as a template, AP1 primer included in the kit, and 1901-21 For primer comprising a 1901-21-specific nucleotide sequence. As a result of subcloning the amplified fragment, followed by sequencing, an approximately 0.7 kb nucleotide sequence comprising the nucleotide sequence of 1901-21 was obtained. Similarly, PCR was performed using AP1 primer included in the kit and B1901-21-specific 1901MR primer. As a result of subcloning the amplified fragment, followed by sequencing, an approximately 0.5 kb nucleotide sequence comprising the nucleotide sequence of 1901-21 was obtained. Thus, this sequence had 1043 bp in total (SEQ ID NO: 2).

| | |
|---|---|
| Primer sequence | |
| 1901-21 For: | |
| AGGAGAGTAACAGTCACAGCAGTAATCA | (SEQ ID NO: 92) |
| 1901 MR: | |
| CCCTGGGCTTTTGTTTCCTCATCT | (SEQ ID NO: 93) |

### EXAMPLE 5

### Elongation of the Genetic Nucleotide Sequence (1913-17)

Using Marathon cDNA Amplification Kit (CLONTECH), PCR was carried out with Human Leukocyte Marathon-Ready cDNA (CLONTECH) as a template, AP1 primer included in the kit, and 1913-17F primer comprising a B1913-specific nucleotide sequence. As a result of subcloning the amplified fragment, followed by sequencing, an approximately 0.5 kb nucleotide sequence comprising the nucleotide sequence of 1913-17 was obtained. Similarly, PCR was performed using AP1 primer included in the kit and 1913-17R primer comprising a 1913-17-specific nucleotide sequence. As a result of subcloning the amplified fragment, followed by sequencing, an approximately 0.4 kb nucleotide sequence comprising the nucleotide sequence of 1913-17 was obtained. Thus, this sequence had 756 bp in total (SEQ ID NO: 3).

| | |
|---|---|
| Primer sequence | |
| 1913-17F: | |
| GGTCAGTTTCCCAACTAAGAGGAGTG | (SEQ ID NO: 94) |
| 1913-17R: | |
| GGAAGTTCTGAGAAAACAGCAGGTG | (SEQ ID NO: 95) |

### EXAMPLE 6

### Elongation of the Genetic Nucleotide Sequence (1852-09)

A biotinylated oligonucleotide was prepared using GENETRAPPER cDNA Positive Selection System (GIBCO BRL), with the oligonucleotide comprising the 1852-09-specific nucleotide sequence (ACATTGGACAAGTGGCACG; SEQ ID NO: 96) as a probe, biotin-14-dCTP included in the kit, and TdT. The YY-1 cDNA library prepared by using TimeSaver cDNA Synthesis Kit (Pharmacia Biotech) was dissociated into single strands using GeneII protein included in the kit and exonuclease III, and then hybridized with the target gene by adding the biotinylated nucleotide. Streptavidin-paramagnetic beads were added thereto, and the clone containing the nucleotide sequence of 1852-09 was obtained by trapping with magnets. Sequencing of the fragment possessed by the selected clone gave a 1931-bp nucleotide sequence (SEQ ID NO: 4) comprising the nucleotide sequence of 1852-09.

### EXAMPLE 7

### Elongation of the Genetic Nucleotide Sequence (1945-03)

Using Marathon cDNA Amplification Kit (CLONTECH), PCR was carried out with Human Leukocyte Marathon-Ready cDNA as a template, AP1 primer included in the kit, and 1945-03 For primer comprising a 1945-03-specific nucleotide sequence. Furthermore, PCR was then carried out with the amplified fragment as a template, using the “AP2” nucleotide sequence in the adaptor and 1945-03 For primer. As a result of subcloning the latter amplified fragment, followed by sequencing, an approximately 2.2 kb nucleotide sequence comprising the nucleotide sequence of 1945-03 was obtained. Similarly, PCR was performed using AP1 primer included in the kit and 1945-03-specific 1945-03 Rev primer. Furthermore, PCR was then carried out with the amplified fragment as a template, by using the “AP2” nucleotide sequence in the adaptor and 1945-03 Rev primer. As a result of subcloning the latter amplified fragment, followed by sequencing, a 1.6 kb nucleotide sequence comprising the nucleotide sequence of B1945 was obtained. Thus, this sequence had 2276 bp in total (SEQ ID NO: 5).

| | |
|---|---|
| Primer sequence | |
| 1945-03 For: | |
| CCATGGAAAATTTGGTCTATCACC | (SEQ ID NO: 97) |
| 1945-03 Rev: | |
| GCTGGAATGAATAAGAAGCTTTGC | (SEQ ID NO: 98) |

### EXAMPLE 8

### Elongation of the Genetic Nucleotide Sequence (1956-04)

PCR was performed with a plasmid comprising the DD sequence of 1956-04 as the template, by using 1956-04 sense and 1956-04 antisense primers comprising 1956-04-specific nucleotide sequences. The amplified fragment was biotinylated using biotin-21-dUTP included in ClonCapture cDNA Selection Kit (CLONTECH), and a complex was formed using this biotinylated probe and RecA protein included in the kit. The biotinylated probe-RecA complex was made to interact with the homologous sequence region of peripheral blood eosinophil cDNA library (double-stranded plasmid library) produced by using SMART cDNA Library Construction Kit (CLONTECH) to form a triple-stranded complex. Magnetic beads carrying immobilized streptavidin was added thereto and the clone containing the nucleotide sequence of 1956-04 was obtained by trapping with magnets. Sequencing of the fragment possessed by the selected clone gave a 293 bp nucleotide sequence (SEQ ID NO: 11) comprising the nucleotide sequence of 1956-04.

| | |
|---|---|
| Primer sequence | |
| 1956-04 sense: | |
| ACAAATCAGAGGTAAAGAGGG | (SEQ ID NO: 99) |
| 1956-04 antisense: | |
| TGATGCATTATTTAGCTCCAG | (SEQ ID NO: 100) |

### EXAMPLE 9

### Change in Gene Expression in Human Peripheral Blood Eosinophils Due to Stimulation by Cytokines

Since eosinophils are considered to be the central inflammatory cells in allergic inflammation, the present inventors examined effects of cytokines on gene expression relating to growth, differentiation, migration and accumulation to a local region, and activation of eosinophils.

The change in gene expression due to cytokine stimulation in eosinophils isolated from 100 ml of peripheral blood from a healthy subject was studied. Isolation of eosinophils was carried out as described above. Eosinophils were plated onto a 24-well plate (1×10⁶ cells/mL). The plate was pre-coated with 1% BSA (immobilizing blocking buffer) at room temperature for 2 hours in order to prevent activation due to adhesion of eosinophils. As cytokines, 0.1, 1, and 10 ng/ml each of interleukin 5 (IL-5), interleukin 4 (IL-4), interferon γ (IFNγ), granulocyte macrophage colony stimulating factor (GM-CSF), and eotaxin were added to each well., and this was cultured for 3 hours in DMEM supplemented with 10% FCS. All of these cytokines are those considered to be related to activation of eosinophils and onset of allergies.

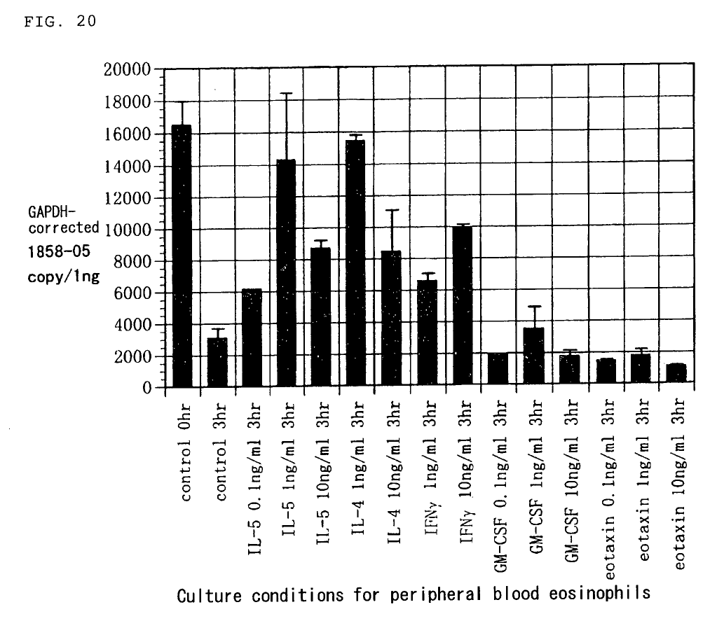

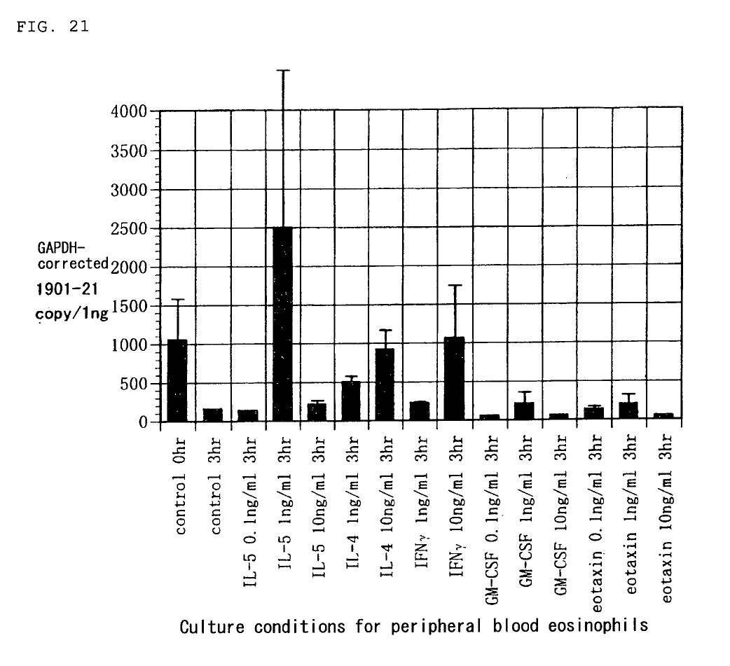

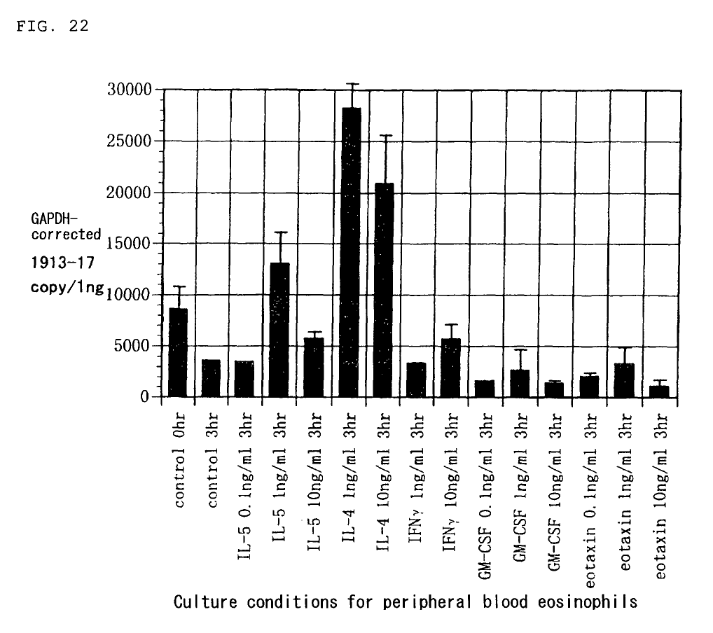

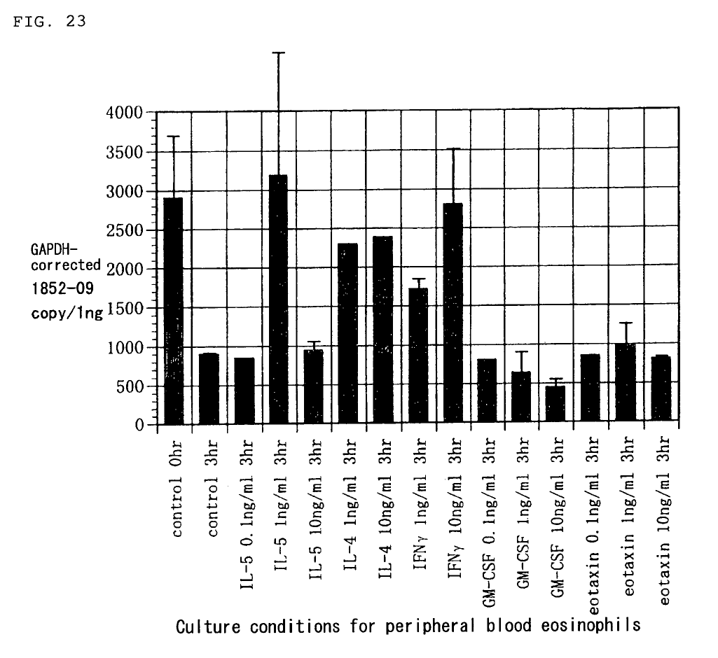

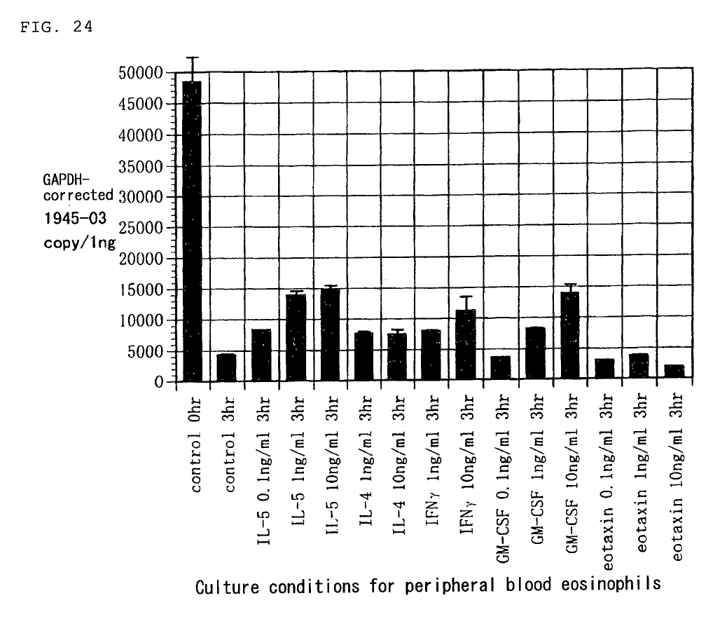

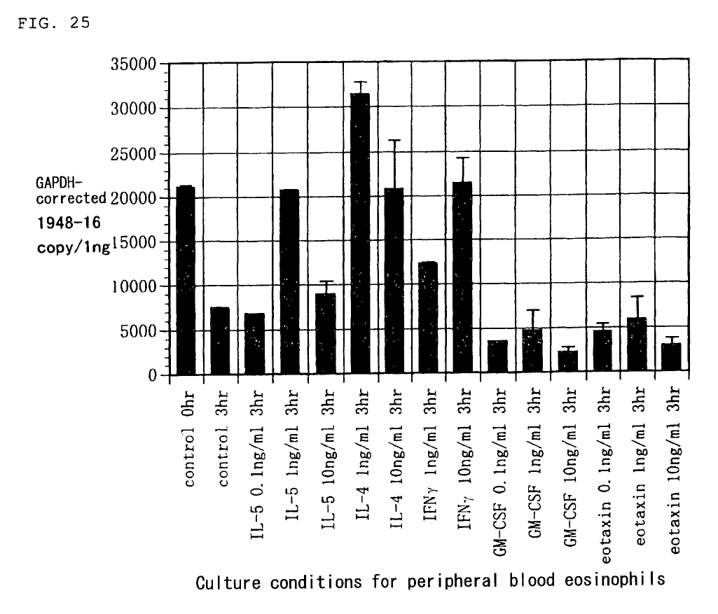

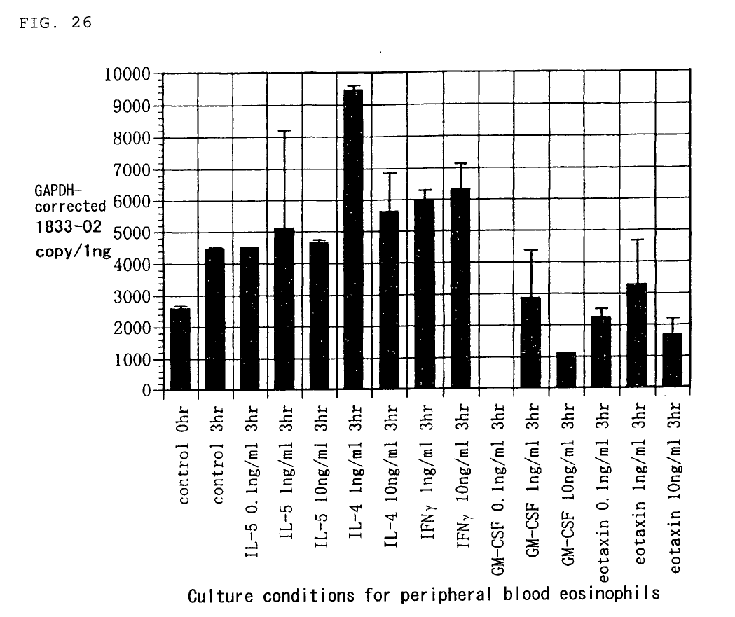

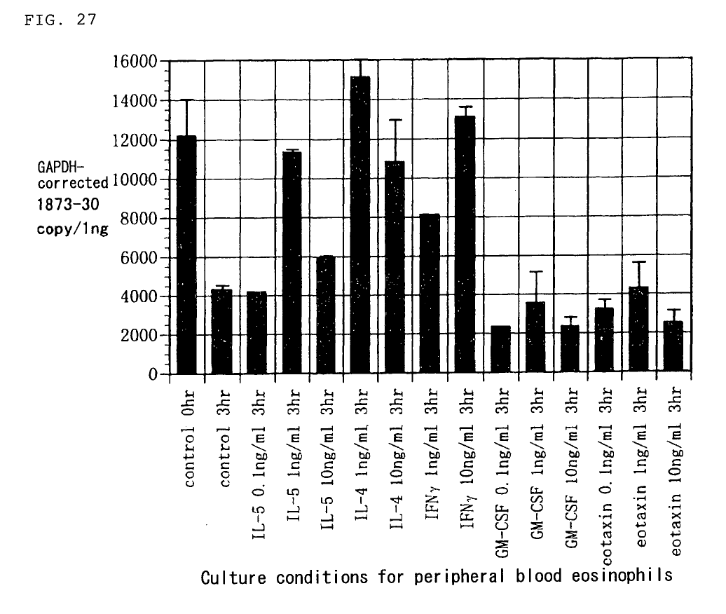

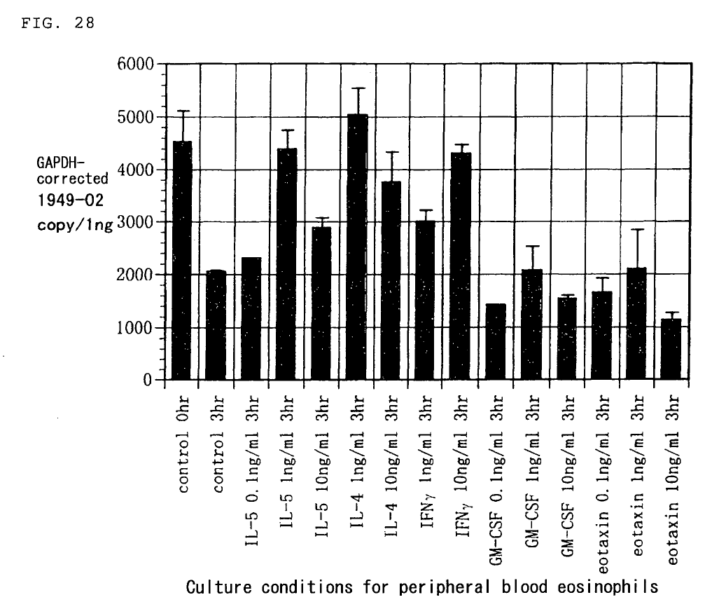

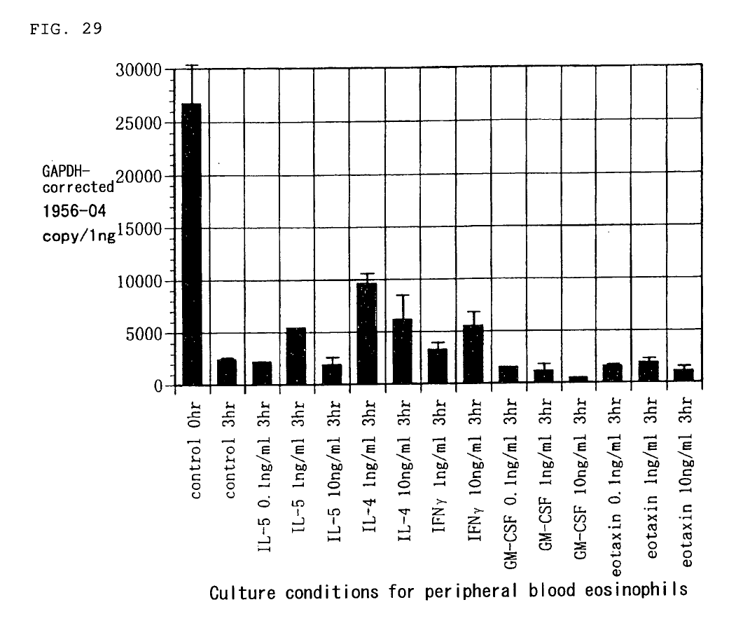

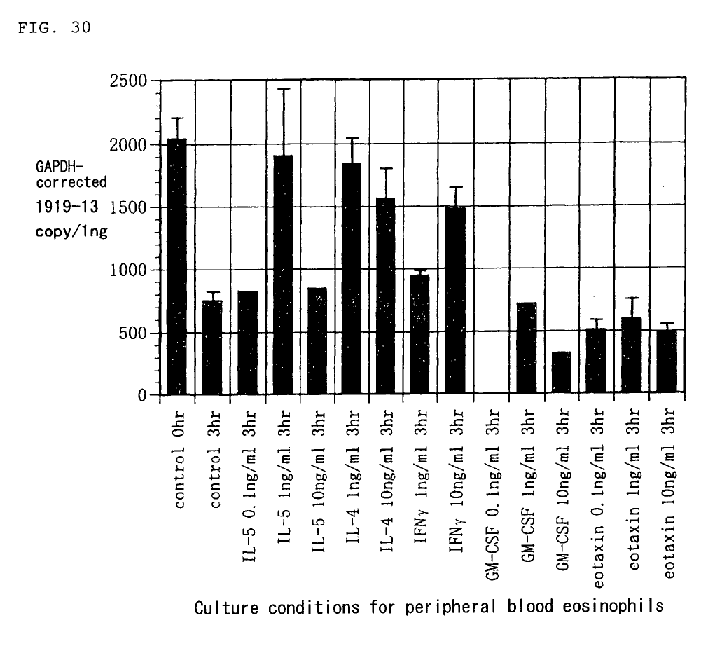

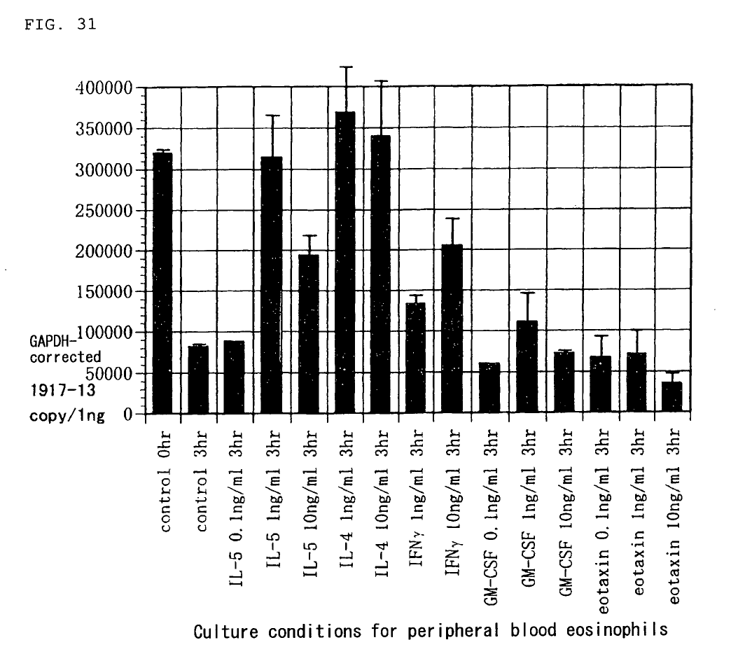

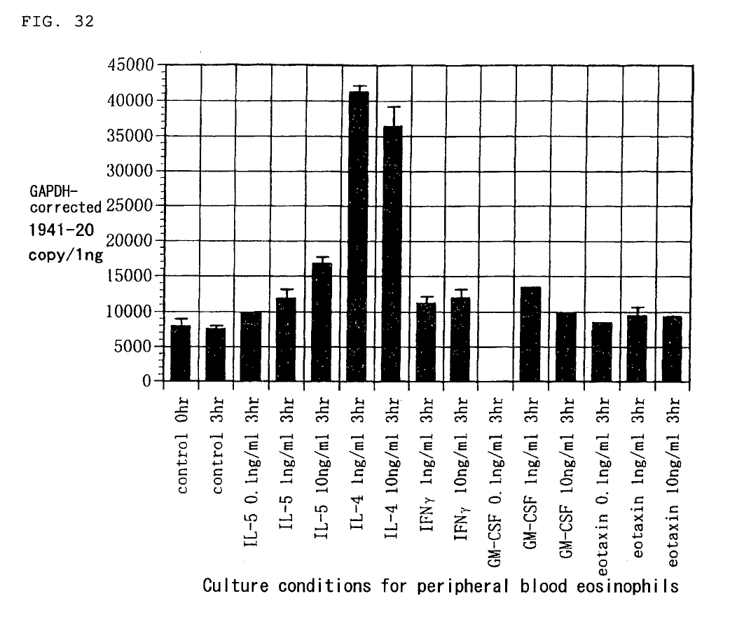

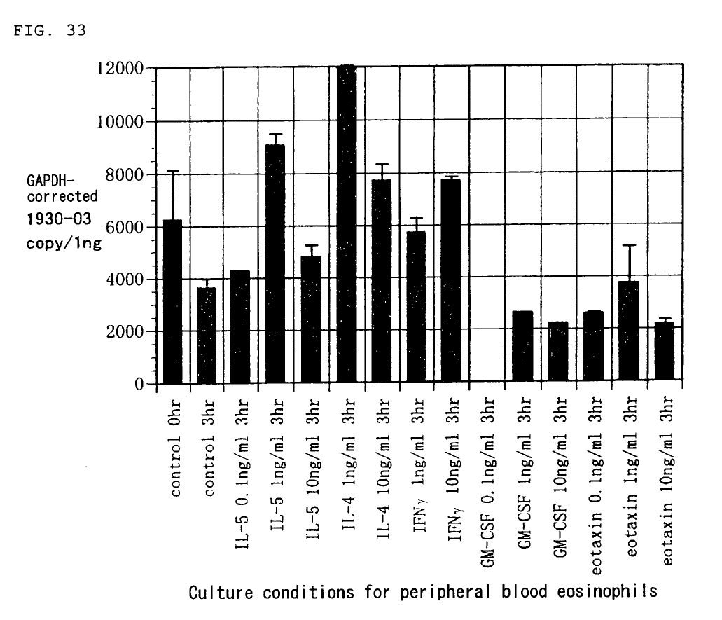

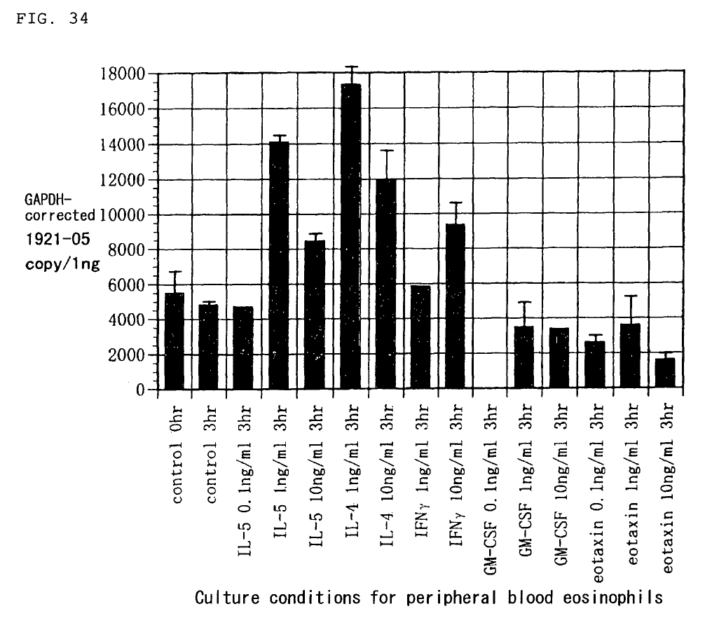

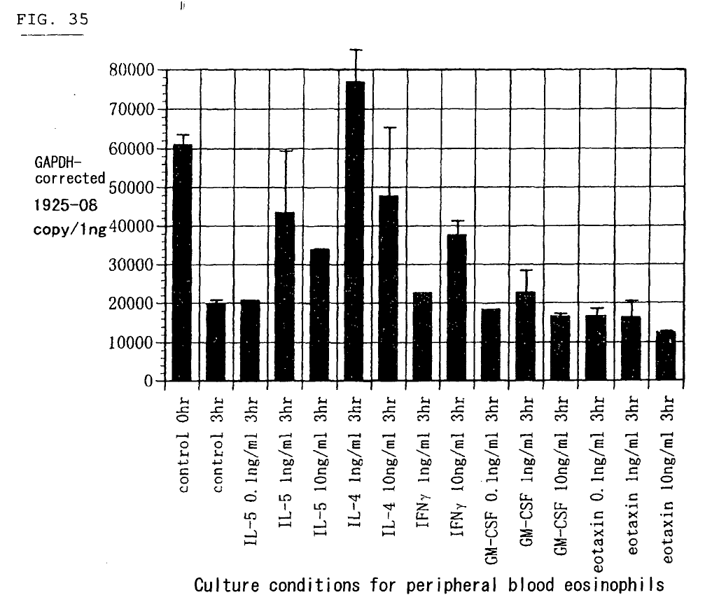

RNAs were prepared in the same manner as in Example 1 for each of the treated eosinophils, and were subjected to gene expression analysis. Expression analysis was carried out on the “1858-05” gene, “1901-21” gene, “1913-17” gene, “1852-09” gene, “1945-03” gene, “1948-16” gene, “1833-02” gene, “1873-30” gene, “1949-02” gene, “1956-04” gene, “1919-13” gene, “1917-13” gene, “1941-20” gene, “1930-03” gene, “1921-05” gene, and “1925-08” gene. The respective primers, probes, and such used were the same as described above. The results obtained for each gene are shown in FIG. 20 to FIG. 35 (all values were corrected with GAPDH for the number of copies per 1 ng of RNA).

Among the cytokines used in the experiment, IL-5 extends the life-time of eosinophils by activating the eosinophils. Therefore, IL-5 treatment increases the expression levels of anti-apoptotic genes, bcl-2 and bax, in eosinophils. Since expression of many of the genes described above increase similarly, as indicated in FIG. 20 to FIG. 35, their expression may be correlated to extension of the life-time of eosinophils, and their relationship to induction and exacerbation of the pathology of allergies was suggested.

Furthermore, the expression of many of the genes described above were also induced by IFNγ, IL-4, and GM-CSF. Regarding these cytokines, there are not many findings relating to gene expression in eosinophils. However, since all are important factors for the onset of allergies, induction of expression of the genes in eosinophils by these cytokines may suggest the possibility that the genes are related to the pathology and exacerbation of allergic diseases.

Genes whose expression was induced by IL-5 include:

“1858-05”, “1913-17”, “1945-03”, “1948-16”, “1833-02”, “1873-30”, “1949-02”, “1956-04”, “1917-13”, “1930-03”, and “1921-05”.

Genes whose expression was induced by IL-4 include:

Increase of expression was observed in all genes except “1945-03”.

Genes whose expression was induced by IFN-γ include:

“1858-05”, “1901-21”, “1852-09”, “1948-16”, “1833-02”, “1873-30”, “1949-02”, “1956-04”, “1919-13”, “1917-13”, “1930-03”, and “1921-05”.

Genes whose expression was induced-by GM-CSF include:

“1945-03”.

### Industrial Applicability

The present invention provides genes that show increased expression in eosinophils of patients with early stage atopic dermatitis. Genes that show elevated expression prior to increase of eosinophils can be utilized as highly sensitive indicators for allergic symptoms. Diagnosis of allergic symptoms at a stage when increase of eosinophils is not observable is normally difficult. However, the indicators provided by the present invention enable early diagnosis that had been difficult with the diagnosis indicators to date. Enabled early diagnosis makes it possible to select accurate therapeutic methods even for early stage allergic diseases.

Increase of eosinophils is an important step in allergic reactions. Thus, genes that show increased expression in eosinophils prior to disease-induced increase in eosinophils are considered to play an important role, especially in the early stages of allergic diseases. Therefore, suppressing the expression and activity of the genes of this invention becomes a strategic target for therapy of allergic diseases, and such genes can be expected to be useful as novel clinical diagnostic indicators for monitoring in such novel therapeutic methods.

Expression levels of indicator genes provided by the present invention can be easily detected regardless of the types of allergens. Therefore, pathological conditions of allergic diseases can be comprehensively understood.

In addition, using peripheral blood eosinophils as a specimen, the expression level of genes can be analyzed in a much less invasive manner to patients according to the method for testing for allergic diseases of the present invention. Furthermore, according to the gene expression analysis method of the present invention, in contrast to protein measurements such as ECP, highly sensitive measurement with a trace sample can be accomplished. Gene analysis technique trends toward high-throughput and lower prices. Therefore, the test method according to the present invention is expected to become an important bedside diagnostic method in the near future. In this sense, these genes associated with pathological conditions are highly valuable in diagnosis.

1. A method of testing for an early stage allergic disease, said method comprising the steps of:
a) measuring the expression level of a gene comprising the nucleotide sequence selected from the group consisting of SEQ ID NOs: 1, 2, 3, 4, 5, 6, 7, 8, 9, 10, 11, 12, 13, 14, 15, 16, and 17 in eosinophil cells of a test subject; and
b) comparing the measured expression level to the expression level of the same gene in eosinophil cells of a healthy subject.

2. The testing method of claim 1, wherein the allergic disease is atopic dermatitis.

3. The testing method of claim 1, wherein the expression level of a gene is measured by cDNA PCR.

4. A reagent for testing for the presence of an early stage allergic disease, said reagent comprising an oligonucleotide that is at least 15 nucleotides long and comprises a nucleotide sequence complementary to a polynucleotide having the nucleotide sequence selected from the group consisting of SEQ ID NOs: 1, 2, 3, 4, 5, 6, 7, 8, 9, 10, 11, 12, 13, 14, 15, 16, and 17 or to its complementary strand.

5. A method of detecting an influence of a candidate compound on the expression level of a polynucleotide of (a) or (b), said method comprising the steps of:
(1) contacting the candidate compound with a cell that expresses a polynucleotide of (a) or (b):
(a) a polynucleotide comprising the nucleotide sequence selected from the group consisting of SEQ ID NOs: 1, 2, 3, 4, 5, 6r 7, 8, 9, 10, 11, 12, 13, 14, 15, 16, and 17;
(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17; and
(2) measuring the expression level of the polynucleotide of (a) or (b) of (1).

6. The method of claim 5, wherein the cell is derived from a leukocyte cell line.

7. A method of detecting an influence of a candidate compound on the expression level of a polynucleotide of (a) or (b):
(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;
(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17;
said method comprising the steps of:
(1) administering the candidate compound to a test animal; and
(2) measuring the expression intensity of the polynucleotide of (a) or (b) in the eosinophil cells of the test animal.

8. A method of screening for a compound that decreases the expression level of the polynucleotide of (a) or (b) above, the method comprising the steps of detecting an influence on the expression level by the method of claim 5 or 7, and selecting a compound that decreases the expression level compared to a control.

9. A method of detecting an influence of a candidate compound on the activity of a transcription regulatory region of a gene comprising the nucleotide sequence selected from the group consisting of SEQ ID NOs: 1, 2, 3, 4, 5, 6, 7, 8, 9, 10, 11, 12, 13, 14, 15, 16, and 17, said method comprising the steps of:
(1) contacting a candidate compound with a cell transfected with a vector comprising the transcription regulatory region of the gene containing the nucleotide sequence selected from the group consisting of-SEQ ID NOs: 1, 2, 3, 4, 5, 6, 7, 8, 9, 10, 11, 12, 13, 14, 15, 16, and 17, and a reporter gene that is expressed under the control of the transcription regulatory region; and
(2) measuring the activity of the reporter gene.

10. A method of screening for a compound that decreases the activity of the transcription regulatory region of a gene containing the nucleotide sequence selected from the group consisting of SEQ ID NOs: 1, 2, 3, 4, 5, 6, 7, 8, 9, 10, 11, 12, 13, 14, 15, 16, and 17, said method comprising the steps of detecting an influence of a candidate compound on the activity by the method of claim 9, and selecting a compound that decreases the activity compared to a control.

11. A vector comprising the transcription regulatory region of a gene containing the nucleotide sequence selected from the group consisting of SEQ ID NOs: 1, 2, 3, 4, 5, 6, 7, 8, 9, 10, 11, 12, 13, 14, 15, 16, and 17, and a reporter gene that is expressed under the control of the transcription regulatory region.

12. A cell carrying the vector of claim 11.

13. A therapeutic agent for an allergic disease, said agent comprising as the active ingredient, a compound obtainable by the method of screening of claim 8 or 10.

14. A therapeutic agent for an allergic disease, which comprises, as a principal ingredient, an antisense DNA against a polynucleotide having the nucleotide sequence selected from the group consisting of SEQ ID NOs: 1, 2, 3, 4, 5, 6, 7, 8, 9, 10, 11, 12, 13, 14, 15, 16, and 17, or a portion thereof.

15. A therapeutic agent for an allergic disease, which comprises, as a principal ingredient, an antibody against a peptide consisting of an amino acid sequence of “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” protein.

16. A polynucleotide of (a) or (b):
(a) a polynucleotide comprising the nucleotide sequence selected from the group consisting of SEQ ID NOs: 1, 2, 3, 4, 5, 6, 7, 8, 9, 10, 11, 12, 13, 14, 15, 16, and 17;
(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17.

17. A protein encoded by the polynucleotide of claim 16.

18. A vector that harbors the polynucleotide of claim 16 in an expressible state.

19. A transformed cell that harbors the polynucleotide of claim 16, or the vector of claim 18.

20. A method of producing the protein of claim 17, said method comprising the steps of culturing the transformed cell of claim 19, and collecting its expression product.

21. An antibody against the-protein of claim 17.

22. A method of immunologically measuring the protein of claim 17, said method comprising the step of observing the immunological reaction between the antibody of claim 21 and the protein of claim 17.

23. An oligonucleotide having at least 15 nucleotides long, and comprising a nucleotide sequence complementary to a polynucleotide having the nucleotide sequence selected from the group consisting of SEQ ID NOs: 1, 2, 3, 4, 5, 6, 7, 8, 9, 10, 11, 12, 13, 14, 15, 16, and 17, or to its complementary strand.

24. A method of measuring the polynucleotide of claim 16, said method comprising the step of observing hybridization of the oligonucleotide of claim 23 to the polynucleotide of claim 16.

25. An early stage allergic disease model animal, wherein said animal is a transgenic non-human vertebrate, in which expression intensity of the polynucleotide of (a) or (b) in eosinophil cells is increased:
(a) a polynucleotide comprising the nucleotide sequence selected from the group consisting of SEQ ID NOs:. 1, 2, 3, 4, 5, 6, 7, 8, 9, 10, 11, 12, 13, 14, 15, 16, and 17;
(b) polynucleotide encoding a protein that shows increased expression in eosinophils of patient with early stage allergic disease, wherein said polynucleotide hybridizes under stringent conditions with a DNA comprising the nucleotide sequence selected from the group consisting of SEQ ID NOs: 1, 2, 3, 4, 5, 6, 7, 8, 9, 10, 11, 12, 13, 14, 15, 16, and 17.

26. A kit for screening for a candidate compound for a therapeutic agent for an allergic disease, said kit comprising cells that express a gene comprising the nucleotide sequence selected from the group consisting of SEQ ID NOs: 1, 2, 3, 4, 5, 6, 7, 8, 9, 10, 11, 12, 13, 14, 15, 16, and 17, and a polynucleotide that is at least 15 nucleotides long and hybridizes to the nucleotide sequence selected from the group consisting of SEQ ID NOs: 1, 2, 3, 4, 5, 6, 7, 8, 9, 10, 11, 12, 13, 14, 15, 16, and 17 or to its complementary strand.

27. A kit for screening for a candidate compound for a therapeutic agent for an allergic disease, said kit comprising cells that express a gene comprising the nucleotide sequence selected from the group consisting of SEQ ID NOs: 1, 2, 3, 4, 5, 6, 7, 8, 9, 10, 11, 12, 13, 14, 15, 16, and 17, and an antibody that recognizes the peptide comprising the amino acid sequence of “1858-05”, “1901-21”, “1913-17”, “1852-09”, “1945-03”, “1948-16”, “1833-02”, “1873-30”, “1937-03”, “1949-02”, “1956-04”, “1919-13”, “1917-03”, “1941-20”, “1930-03”, “1921-05”, or “1925-08” protein.
